(19) United States
(12) Patent Application Publication
(10) Pub. No.: US 2025/0387381 A1
(43) Pub. Date: Dec. 25, 2025

(54) COMPOSITIONS AND METHODS FOR ENHANCED DRUG LOADING OF LONG-ACTING IN SITU FORMING IMPLANTS AND USES THEREOF

(71) Applicant: The University of North Carolina at Chapel Hill, Chapel Hill, NC (US)

(72) Inventors: J. Victor GARCIA-MARTINEZ, Durham, NC (US); Martina Kovarova, Chapel Hill, NC (US); Miriam Braunstein, Chapel Hill, NC (US); Manse Kim, Chapel Hill, NC (US); Claire Post, Chapel Hill, NC (US)

(21) Appl. No.: 18/836,156

(22) PCT Filed: Feb. 13, 2023
(86) PCT No.: PCT/US2023/012930

§ 371 Date: Aug. 6, 2024

Related U.S. Application Data
(60) Provisional application No. 63/309,151, filed on Feb. 11, 2022.

(51) Int. Cl. A61K 31/438; A61K 9/00; A61K 47/34; A61P 31/06

(52) U.S. Cl. CPC A61K 31/438; A61K 9/0024; A61K 47/34; A61P 31/06

(57) ABSTRACT

Extended release or long-acting injectable compositions for use as in-situ forming implants are provided. The extended release injectable compositions and resulting long-acting in-situ forming implants include one or more drugs or active agents, a biocompatible solvent, a biodegradable polymer, and either an amphiphilic additive or a hydrophobic additive, or a combination of an amphiphilic additive and a hydrophobic additive. Such compositions are made to be injected in subjects in need of treatment to form in-situ formed implants within the subject, such in-situ formed implants having increased drug load and improved drug release for a longer duration. Methods of using such compositions, and treating subjects therewith are provided. Methods of making such compositions are also provided.

### CROSS-REFERENCE TO RELATED APPLICATIONS

This application is a national phase claiming the benefit of and priority to International Patent Application No. PCT/US2023/012930, filed on Feb. 13, 2023, which claims the benefit of priority of U.S. Provisional Patent Application Ser. No. 63/309,151, filed Feb. 11, 2022, the contents of which are hereby incorporated by reference in their entirety.

### GRANT STATEMENT

This invention was made with government support under Grant Numbers AI111899, A1123010, and CA016086 awarded by the National Institutes of Health. The government has certain rights in the invention.

### TECHNICAL FIELD

Provided herein are compositions of long-acting in situ forming implants with enhanced drug loading. Provided are also methods for making enhanced drug loading long-acting in situ forming implants, and uses of the same for treating conditions in subjects.

### BACKGROUND

Long-acting (LA) delivery systems are one of the most important approaches for improving adherence to treatments that require consistent, long-term drug administration. To be effective, the material composition of LA formulations should allow for sufficient drug release for prolonged periods of time after their administration. The hybrid nature of injectable in situ forming implant (ISFI) LA formulations requires a delicate composition balance to accommodate sufficient amounts of polymer and biocompatible solvent. This severely limits the amount, number, and types of drugs that can be successfully formulated. What is needed are improved formulations, compositions and methods for LA delivery systems. Such advancements are provided herein.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

Provided in some embodiments are extended release or long-acting injectable compositions, the compositions comprising a drug or active agent; a biocompatible solvent; a biodegradable polymer; and an amphiphilic additive, or a hydrophobic additive, or a combination of an amphiphilic additive and a hydrophobic additive. In some embodiments, In some embodiments, the extended release or long-acting injectable composition provides a sustained release of the drug or active agent upon administration to a subject in vivo or upon formation of an in-situ forming implant in vitro.

In some embodiments, the composition comprises the amphiphilic additive. In some embodiments, the amphiphilic additive is selected from the group consisting of Kolliphor®HS 15, Kolliphor® RH40, Kolliphor® EL, Tween 80, Tween 20, Vitamin E TPGS, Polysorbate 40, Polysorbate 60, Poloxamer 124, Poloxamer 188, Poloxamer 338, Poloxamer 407, Poloxamer 105, Poloxamer 238, Poloxamer 331, Poloxamer 334, Poloxamer 335, PEG, Span 20, Span 40, Span 80, Span 60, Triton X-100.

In some embodiments, the composition comprises the hydrophobic additive, optionally wherein the hydrophobic additive increases release of the drug or active agent from a solidified implant formed from the extended release or long-acting injectable composition by at least 5%, optionally by about 20% to about 150%, as compared to a solidified implant without a hydrophobic additive. In some embodiments, the hydrophobic additive comprises a saturated fatty acid, a monounsaturated fatty acid, a polyunsaturated fatty acid, and/or a combination thereof, optionally wherein the saturated fatty acid, monounsaturated fatty acid, polyunsaturated fatty acid, and/or combination thereof comprises oleic acid, stearic acid, arachidic acid, palmitic acid, linolic acid, myristic acid, α- or β-eleostearic acid, 9,11-octadecadienoic acid, and/or eicosapentaenoic acid. In some embodiments, the hydrophobic additive comprises a fatty alcohol, optionally wherein the fatty alcohol comprises hexacosanol, octacostanol, dotriacontanol, and/or combinations thereof. In some embodiments, the hydrophobic additive comprises a terpene, optionally wherein the terpene comprises nerolidol, farnesol, and/or combinations thereof. In some embodiments, the hydrophobic additive comprises a sterols, optionally wherein the sterol comprises cholesterol, sitosterol, stigmasterol, stigmastanol, and/or combinations thereof. In some embodiments, the hydrophobic additive comprises a tocopherol, optionally wherein the tocopherol comprises vitamin E, Vitamin E derivatives, and/or combinations thereof.

In some embodiments, the drug or active agent is selected from the group consisting of: Analgesics, Antianxiety Drugs, Antiarrhythmics, Antibacterials, Antibiotics, Anticoagulants and Thrombolytics, Anticonvulsants, Antidepressants, Antiemetics, Antifungals, Antihistamines, Antihypertensives, Anti-Inflammatories, Antineoplastics, Antipsychotics, Antipyretics, Antivirals, Barbiturates, Beta-Blockers, Bronchodilators, Corticosteroids, Cytotoxics, Diuretics, Hormones, Hypoglycemics, Immunosuppressives, Muscle Relaxants, Sedatives, Tranquilizer, and Vitamins. In some embodiments, the drug or active agent is selected from the group consisting of: Bedaquiline, Delaminid, Clofazamine, Rifapentine, cilastatin, Moxifloxacin, Rifabutin, Terizidone, Prothionamide, Ethionamide, Pretamonid, Rifampin (RIF), Levofloxacin, Linezolid, Capreomycin, Para-aminosalicylic acid (PAS), Ethambutol (EMB), Pyrazinamide (PZA), Imipenem, Kanamycin, Ioniazid (INH), Amikacin, Cycloserine, Streptomycin, Meropenem (Mpm), Rifabutin, Cefoxitin, Clarithromycin, Tigecycline, Azithromycin, Minocycline, Apramycin, Isoniazid. In some embodiments, the composition comprises a combination of more than one drug or active agent.

In some embodiments, the biocompatible solvent is selection from one or more of Dimethyl sulfoxide (DMSO), n-Methyl pyrrolidone (NMP), benzyl alcohol (BA), benzyl benzoate (BB) or combinations thereof. In some embodiments, the biocompatible solvent comprises a cosolvent system using NMP and DMSO, optionally wherein the DMSO:NMP ratio of about 1:99 to about 50:50 (e.g. when bedaquiline is the active agent), optionally wherein the DMSO:NMP ratio of about 1:99 to about 99:1 (e.g. when rifabutin is the active agent). In some embodiments, the biodegradable polymer comprises a low molecular weight (MW) polymer, e.g. MW less than about 25 Da, optionally less than about 150 Da.

In some embodiments, the biodegradable polymer comprises a range of lactic acid:glycolic acid ratios of about 50:50 to about 95:5. In some embodiments, the biodegradable polymer comprises a biodegradable poly(lactic-co5 glycolic-acid) (PLGA), i.e. a polymer with molecular weight of about 5 kDA to about 30 kDa and lactic acid:glycolic acid ratio of about 50:50).

In some embodiments, the extended release or long acting injectable composition is configured as an in-situ forming implant (ISFI), optionally wherein the extended release or long acting injectable composition comprises a liquid formulation that is configured to be injectable in a subject, optionally wherein the extended release or long acting injectable composition is injectable subcutaneously. In some embodiments, the extended release comprises a substantially sustained release of the drug or active agent over weeks or months, optionally at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 15 weeks, at least about 20 weeks, at least about 30 weeks, or more.

Provided herein in some embodiments are extended release or long-acting injectable compositions, wherein the composition comprises: a biodegradable poly(lactic-co5 glycolic-acid) (PLGA), optionally wherein the PLGA is a polymer with molecular weight 10.6 kDa and lactic acid:glycolic acid ratio 50:50; a biocompatible water miscible solvent; an amphiphilic additive (e.g. Kolliphor HS15); a hydrophobic additive (e.g. oleic acid); and an active ingredient(s) or combination thereof selected from rifabutin (RFB), rifapentine, or rifampin, or any drug or drug combination for treating Nontuberculous Mycobacteria (NTM) and/or tuberculosis. In some embodiments, the composition is suitable for treatment, prevention and/or amelioration of symptoms of Nontuberculous Mycobacteria (NTM) and/or tuberculosis in a subject.

In some embodiments, the compositions are configured to be administered to a subject in need of treatment as an in-situ forming implant (ISFI) about once a month, optionally about once every two, three, four, five or six months. In some embodiments, a concentration or quantity of the drug or active agent in the composition is increased by about 200% to about 350% as compared to a composition not having an amphiphilic additive.

Provided herein are methods of treating a subject, the methods comprising administering to a subject in need of treatment an extended release or long-acting injectable composition as disclosed herein, wherein administration of the extended release or long-acting injectable composition to the subject forms a long-acting in-situ forming implant (LA ISFI) in the subject, wherein the subject is treated. In some embodiments, the LA ISFI in the subject provides to the subject a drug or active agent for weeks or months, optionally at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 15 weeks, at least about 20 weeks, at least about 30 weeks, or more. In some embodiments, the drug or active agent provided to the subject from the LA ISFI is selected from the group consisting of. Analgesics, Antianxiety Drugs, Antiarrhythmics, Antibacterials, Antibiotics, Anticoagulants and Thrombolytics, Anticonvulsants, Antidepressants, Antiemetics, Antifungals, Antihistamines, Antihypertensives, Anti-Inflammatories, Antineoplastics, Antipsychotics, Antipyretics, Antivirals, Barbiturates, Beta-Blockers, Bronchodilators, Corticosteroids, Cytotoxics, Diuretics, Hormones, Hypoglycemics, Immunosuppressives, Muscle Relaxants, Sedatives, Tranquilizer, and Vitamins.

In some embodiments, the subject is a mammal, optionally a human. In some embodiments, the subject is suffering from an infectious disease, optionally wherein the subject is suffering from a bacterial infection (e.g. Mycobacterium tuberculosis, non-tuberculosis Mycobacterium, Helicobacter (pylori), Acinetobacter and Staphylococcus bacteria), optionally wherein the subject is suffering from a viral infection (e.g. HIV, Hepatitis viruses (including hep C), tuberculosis, malaria.

Provided are methods of treating Nontuberculous Mycobacteria (NTM) and/or tuberculosis in a subject, the methods comprising administering to a subject in need of treatment an extended release or long-acting injectable composition as disclosed herein, wherein the NTM or tuberculosis is substantially or completely treated. In some embodiments, administration of the extended release or long-acting injectable composition to the subject forms a long-acting in-situ forming implant (LA ISFI) in the subject, optionally wherein administration comprises subcutaneous injection of the extended release or long-acting injectable composition to the subject. In some embodiments, the treatment provided to the subject by the LA ISFI treats the NTM or tuberculosis for weeks or months, optionally at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 15 weeks, at least about 20 weeks, at least about 30 weeks, or more. In some embodiments, the drug or active agent provided to the subject from the LA ISFI to treat the NTM or tuberculosis is selected from the group consisting of: the group consisting of: Bedaquiline, Delaminid, Clofazamine, Rifapentine, cilastatin, Moxifloxacin, Rifabutin, Terizidone, Prothionamide, Ethionamide, Pretamonid, Rifampin (RIF), Levofloxacin, Linezolid, Capreomycin, Para-aminosalicylic acid (PAS), Ethambutol (EMB), Pyrazinamide (PZA), Imipenem, Kanamycin, Ioniazid (INH), Amikacin, Cycloserine, Streptomycin, Meropenem (Mpm), Rifabutin, Cefoxitin, Clarithromycin, Tigecycline, Azithromycin, Minocycline, Apramycin, Isoniazid. In some embodiments, the subject is a mammal, optionally a human.

Provided in some aspects are methods of making an extended release or long-acting injectable composition, the method comprising: solubilizing a biodegradable polymer in a biocompatible solvent containing an amphiphilic additive at a ratio of about 2:1 biocompatible solvent:biodegradable polymer to about 6:1 biocompatible solvent:biodegradable polymer, optionally about 4:1 biocompatible solvent:biodegradable polymer; and adding a drug or active agent to the composition, wherein the drug or active agent is added at a concentration ranging from about 100 mg mL−1 to about 500 mg mL−1, optionally about 200 mg mL−1 to about 400 mg mL−1. In some embodiments, the method further comprises adding a hydrophobic additive to the composition. In some embodiments, the hydrophobic additive comprises a saturated fatty acid, a monounsaturated fatty acid, a polyunsaturated fatty acid, and/or a combination thereof, optionally wherein the saturated fatty acid, monounsaturated fatty acid, polyunsaturated fatty acid, and/or combination thereof comprises oleic acid, stearic acid, arachidic acid, palmitic acid, linolic acid, myristic acid, α- or β-eleostearic acid, 9,11-octadecadienoic acid, and/or eicosapentaenoic acid. In some embodiments, the hydrophobic additive comprises a fatty alcohol, optionally wherein the fatty alcohol comprises hexacosanol, octacostanol, dotriacontanol, and/or combinations thereof. In some embodiments, the hydrophobic additive comprises a terpene, optionally wherein the terpene comprises nerolidol, farnesol, and/or combinations thereof. In some embodiments, the hydrophobic additive comprises a sterols, optionally wherein the sterol comprises cholesterol, sitosterol, stigmasterol, stigmastanol, and/or combinations thereof. In some embodiments, the hydrophobic additive comprises a tocopherol, optionally wherein the tocopherol comprises vitamin E, Vitamin E derivatives, and/or combinations thereof.

In some embodiments, the drug or active agent is selected from the group consisting of: Analgesics, Antianxiety Drugs, Antiarrhythmics, Antibacterials, Antibiotics, Anticoagulants and Thrombolytics, Anticonvulsants, Antidepressants, Antiemetics, Antifungals, Antihistamines, Antihypertensives, Anti-Inflammatories, Antineoplastics, Antipsychotics, Antipyretics, Antivirals, Barbiturates, Beta-Blockers, Bronchodilators, Corticosteroids, Cytotoxics, Diuretics, Hormones, Hypoglycemics, Immunosuppressives, Muscle Relaxants, Sedatives, Tranquilizer, and Vitamins. In some embodiments, the drug or active agent is selected from the group consisting of: Bedaquiline, Delaminid, Clofazamine, Rifapentine, cilastatin, Moxifloxacin, Rifabutin, Terizidone, Prothionamide, Ethionamide, Pretamonid, Rifampin (RIF), Levofloxacin, Linezolid, Capreomycin, Para-aminosalicylic acid (PAS), Ethambutol (EMB), Pyrazinamide (PZA), Imipenem, Kanamycin, Ioniazid (INH), Amikacin, Cycloserine, Streptomycin, Meropenem (Mpm), Rifabutin, Cefoxitin, Clarithromycin, Tigecycline, Azithromycin, Minocycline, Apramycin, Isoniazid. In some embodiments, the composition comprises a combination of more than one drug or active agent.

In some embodiments, the biocompatible solvent is selection from one or more of Dimethyl sulfoxide (DMSO) and n-Methyl pyrrolidone (NMP), or combinations thereof. In some embodiments, the biocompatible solvent comprises a cosolvent system using NMP and DMSO, optionally wherein the DMSO:NMP ratio of about 1:99 to about 50:50 (e.g. when bedaquiline is the active agent), optionally wherein the DMSO:NMP ratio of about 1:99 to about 99:1 (e.g. when rifabutin is the active agent). In some embodiments, the biodegradable polymer comprises a low molecular weight (MW) polymer, e.g. MW<25 Da. In some embodiments, the biodegradable polymer comprises a range of lactic acid:glycolic acid ratios of about 50:50 to about 95:5. In some embodiments, the biodegradable polymer comprises a biodegradable poly(lactic-co5 glycolic-acid) (PLGA), i.e. a polymer with molecular weight of about 10.6 kDa and lactic acid:glycolic acid ratio of about 50:50). In some embodiments, addition of the amphiphilic additive increases the drug or active agent load in the composition by about 200% to about 350% as compared to a composition not having an amphiphilic additive.

Accordingly, objects of the presently disclosed subject matter having been stated above, other objects and advantages of the presently disclosed subject matter will become apparent to those skilled in the art after a study of the following description, Drawings and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIG. 1 is a schematic illustration showing significant and surprising changes in the material composition of in situ forming implant formulations that result in structural changes, unexpectedly increased payload, reduced erosion, and surprising improvement in long-term effective drug delivery. FIG. 1A: Schematic showing long-acting (LA) in situ forming implant (ISFI) formulation, using LA rifabutin (RFB) as an example, composition consisting of poly(lactic-co-glycolic-acid (PLGA) as the biodegradable polymer, dimethyl sulfoxide (DMSO) or N-Methyl-2-pyrrolidone (NMP) as a biocompatible solvent, RFB as an example active pharmacological ingredient, and Kolliphor®HS 15 as an example additive (each specific component for example only, but not meant to be limiting). FIG. 1B: The liquid formulation is injectable and can be administered subcutaneously. FIG. 1C: The range of the drug load surprisingly increases in LA-RFB formulations after addition of amphiphilic additives. FIG. 1D: A solidified implant of 50 μL LA-RFB, scale bar is 5 mm. FIG. 1E: Microphotographs of implants without additives (left panel) and with additives (right panel). Scale bar is 2 μm. FIG. 1F: Formalin-fixed whole lung lobes of mice treated with placebo (upper panel) or LA-RFB (lower panel) prior to Mycobacterium tuberculosis (Mtb) exposure. White lesions caused by Mtb are visible in lungs from mice which received placebo and are not present in mice which received LA-RFB.

FIGS. 2A-2D: In vitro release properties of LA-ISFI, e.g. LA-RFB, depend on polymer and solvent composition. LA-RFB formulations were evaluated under sink conditions after injecting 30 μL of the formulation into release medium (PBS). A) Cumulative release of RFB from the formulation (RFB9) as a percent of the initially injected RFB. Dotted line indicates initial burst, the cumulative RFB released within the first 72 h post injection. B) RFB daily release (μg day⁻¹) over time. C) RFB daily release (ug day⁻¹) from LA-RFB formulations with polymers of different MW and indicated solvent: polymer ratios four weeks post injection. R1-12 indicate formulations RFB1-12 (Table 3). D) RFB daily release from LA-RFB with 10.6 kDa PLGA and different solvents at four weeks of incubation. R1-12 indicates formulations RFB1-12 (Table 3). For panels A-D n=3, in A,B means and individual values are shown, in C,D means±SD are shown. * P<0.05, ** P<0.01, ***P<0.001, **** P<0.0001, statistical significance in panel C and D was determined using a one-way ANOVA.

FIGS. 3A-3D: Amphiphilic additives increase the saturated solubility of the active agent, e.g. RFB, in organic biocompatible solvents. A) The saturated solubility of RFB in NMP in the presence of indicated concentrations of Kolliphor®HS 15. B-D) The saturated solubility of RFB in DMSO in presence of following additives: TPGS (B), Tween 80 or Tween 20 (C), and Pluronic F127 or Pluronic F68 (D). (n=3 per concentration, means and individual measurements are shown.) Dashed line indicates the solubility of RFB in solvent without additives.

FIGS. 4A-F: Amphiphilic additives increase drug load in LA-ISFI formulations and extend the duration of drug release. A) Saturated solubility of RFB in DMSO with different concentrations of Kolliphor®HS 15 (n=3 per concentration indicated in the log x axis). Dashed line indicates RFB solubility in DMSO without the addition of Kolliphor®HS 15. B) Comparison of drug loads in LA-RFB formulations with or without Kolliphor®HS 15 (RFB9Sol, RFB11Sol, RFB13Sol and RFB9, RFB11, RFB13, respectively); n=3 per formulation. Mean±SD is shown. C) Cumulative in vitro RFB release (%) from ISFI formulations containing Kolliphor®HS 15; n=3 per formulation; mean and individual values are shown. D) Comparison of in vitro release bursts between LA-RFB formulations with and without Kolliphor®HS 15 (n=3 per group, mean±SD). E) In vitro cumulative RFB release (μg) from LA-RFB ISFI formulations with and without Kolliphor®HS 15; left: RFB9 vs. RFB9Sol, middle: RFB11 vs. RFB11Sol, right: RFB13 vs. RFB13Sol. Mean and individual values are shown, n=3 per formulation. F) Comparison of RFB plasma concentration after a single subcutaneous injection (50 L) into BALB/c mice of LA-RFB ISFI formulations with or without Kolliphor®HS 15 (RFB9 vs. RFB9Sol in the left panel, RFB11 vs. RFB11Sol in the middle panel, RFB13 vs. RFB13Sol in the right panel. n=5 (RFB9, RFB11, RFB13, RFB9Sol), n=3 (RFB11Sol), and n=2 (RFB13Sol); mean and individual values are shown. * P<0.05, ** P<0.01, **** P<0.0001, statistical significance in panel B and D was determined using an unpaired t-test.

FIGS. 5A-5E: Improved drug/active agent, e.g. RFB, release by formulations containing uncapped acid-ending PLGA. A) Daily in vitro RFB release from LA-RFB formulations containing acid-ending PLGA (LA:GA 50:50, MW 13.5 kDa) without Kolliphor®HS 15 (RFB14) or with Kolliphor®HS 15 (RFB14Sol), n=3 per formulation. B) In vivo RFB plasma concentrations in BALB/c after a single subcutaneous injection (50 L) of RFB14 or RFB14Sol (n=4). The dotted line indicates RFB MIC. C) RFB tissue concentrations at two and six weeks post administration of RFB14Sol (50 μL) in BALB/c mice; n=4 per time point, mean±S.E.M. D) Tissue to plasma ratio of RFB concentrations at two and six weeks post administration of RFB14Sol (n=4 per time point, mean±S.E.M.). Spl: spleen, Kid: kidney, LN: lymph nodes. E) Pharmacokinetics of RFB after a single subcutaneous administration of LA-RFB (50 μL) in BALB/c mice followed by a second injection of RFB14Sol (50 μL) at 8 weeks (n=4) or 12 weeks (n=2) after the first dose. Mean±S.E.M are shown; arrows indicate the time point of the booster injections, and the dotted line indicates RFB MIC.

FIGS. 6A-6C: RFB stability in RFB14Sol formulation. RFB14Sol was stored at room temperature (25° C.) in the dark for 18 months. A) Relative RFB amounts in the formulation over time (n=3). B,C) Normalized HPLC histograms of RFB in RFB14Sol formulation (B) or RFB in DMSO without polymer (C) for 6 months.

FIGS. 7A-7E: Impact of Kolliphor®HS 15 and increased drug load on drug, e.g. RFB, release kinetics from ISFI formulations. A-D) In vitro evaluation of formulations RFB9 (127 mg g⁻¹ RFB) and RFB14 (130 mg g⁻¹ RFB) with formulations with high drug load and 0.45 wt % Kolliphor®HS 15 (RFB9Sol: 293 mg g⁻¹ RFB, RFB14Sol: 294 mg g⁻¹ RFB), and formulations containing Kolliphor®HS 15 and the same drug load as RFB9 and RFB14 (RFB9Sol=9: 128 mg g⁻¹ RFB, RFB14Sol=14: 132 mg g⁻¹ RFB) (n=3 per formulation). Cumulative RFB release in % over time of RFB9, RFB9Sol, RFB9Sol=9 (A) and RFB14, RFB14Sol, RFB14Sol=14 (C). Cumulative RFB release (g) over time of RFB9, RFB9Sol, RFB9Sol=9 (B) and RFB14, RFB14Sol, RFB14Sol=14 (D). E) Representative images of a placebo implant of with 0.45 wt % Kolliphor®HS 15 but without RFB (placebo14Sol), an implant with 132 mg g⁻¹ RFB load and Kolliphor®HS 15 (RFB14Sol=14), and an implant with 294 mg g⁻¹ RFB load and Kolliphor®HS 15 (RFB14Sol) in PBS (pH 7.4) over time. Scale bar=1 cm. n=3 implants per formulation.

FIGS. 8A-8C: Surface morphology of LA-ISFI, e.g. LA-RFB, implants depends on implant composition. A) Representative SEM images of the surface of implants formed by RFB14, RFB14Sol, and RFB14Sol=14 and their corresponding placebos (placebo14 and placebo14Sol) in PBS after 3, 7, and 28 days of incubation. Scale bar=10 μm. B-C) Quantitative SEM analysis of implant surface images for changes in porous area (B) and pore size (C) over time. * P<0.05, ** P<0.01, ***P<0.001, **** P<0.0001 (one-way ANOVA).

FIGS. 9A-9B: Structure of LA-ISFI, e.g. LA-RFB, implants. A,B) SEM images of the surface (A, scale bar=2 μm,) and the internal structures (B, scale bar=10 μm) of the implants formed by RFB14, RFB14Sol, RFB14Sol=14, and the corresponding placebos (placebo14 and placebo14Sol) over time (for the surface: 3, 7, and 28 days of incubation, for the internal structure: 7 and 28 days of incubation) (I=inside area of implant and S=shell of implant).

FIGS. 10A-10G: In vivo analysis of the efficacy of RFB14Sol against Mycobacterium tuberculosis infection. A) Experimental schema for pre-exposure prophylaxis efficacy testing of LA-RFB. BALB/c mice were untreated (n=4) or treated with a single subcutaneous injection of placebo (n=6) or RFB14Sol (n=6) formulations. Two weeks after treatment, mice were exposed to Mtb. Control untreated mice were used to determine the dose of exposure 24 h after infection (n=4). ISFI treated mice were analyzed for Mtb infection four weeks post-exposure. B) Bacterial burden in lung, liver, and spleen of placebo and RFB14Sol treated mice four weeks after Mtb exposure. C) H&E staining of lung sections shows pathological changes in mice treated with placebo but not in mice treated with RFB14Sol. D) Experimental schema for post-exposure LA-RFB prophylaxis. BALB/c mice (n=22) were exposed to Mtb; 24 hours later, a group of 4 was analyzed to determine the exposure dose. Mtb infection was assessed again one-week post exposure in a group of 6 mice to determine the levels of infection. At this time the remaining infected mice were treated with placebo (n=6) or RFB14Sol (n=6). Animals were harvested and analyzed for Mtb infection three weeks later (four weeks post exposure to Mtb). E) Bacterial burden in mice 7 days post Mtb exposure. F) Bacterial burden four weeks post Mtb exposure. G) H&E staining of lung sections show pathological changes in mice treated with placebo but not in mice treated with RFB14Sol. Scale bars in H&E images are 300 μm in low magnification images and 50 μm in higher magnification images. Dotted lines in panels B, E, and F indicate the limit of detection. Arrows in panels C and G indicate lesions caused by Mtb. ** P<0.01, significance determined using a Mann-Whitney U test for each organ.

FIGS. 11A-11C: In vivo Mtb exposure dose and LA-RFB treated lungs. A) Exposure dose for the pre-exposure prophylaxis experiment. Dose is displayed as CFU (left) and CFU g⁻¹ of lung tissue (right). Data is expressed as mean±S.E.M. B) Representative H&E images of the lung of an uninfected BALB/c mouse treated with 50 μL RFB14Sol. Mice were necropsied 4 weeks after treatment, n=4. Scale bars in low magnification images are 300 μm and 50 m in higher magnification images. C) Exposure dose for the post-exposure treatment experiment. Dose is expressed as CFU (left) and CFU g⁻¹ of lung tissue (right). Data is expressed as mean±S.E.M.

FIGS. 12A-12B: Pharmacokinetic profile of RFB-ISFI Formulations in BALB/c mice. A) Mice were injected 5 μl of RFB-ISFI formulations F1 (294±5) mg/g, n=4 or F2 (402±6) mg/g, n=5. RFB plasma concentration was measured until it reached minimal inhibitory concentration (MIC, 64 ng/ml). B) Rifabutin released measured as area under the time-plasma concentration curve (AUC). Mean values and individual replicates are shown; n=4 for F1 and n=5 for F2. Differences were evaluated using a Mann-Whitney U test.

FIGS. 13A-13B: Hydrophobic additives increase RFB release in vivo. A) Pharmacokinetic profile of formulation F4 (n=4) and its comparison to formulations F1 (n=4) and F2 (n=5). B) RFB released during the first 10 weeks post administration of indicated formulations expressed as an area under the time-plasma RFB concentration curve (AUC). Mean values and individual replicates are shown. Differences were evaluated using one-way ANOVA with the Fisher's LSD multiple comparison test.

FIG. 14A-14H: Terminal sterilization by autoclaving of RFB-ISFI formulations. Formulations F2 and F4 were autoclaved 121° C. for 20 min and the properties of autoclaved formulations (marked as (A)) were compared to non-autoclaved formulations (marked as (NA)). Indicated formulations were injected into release medium (2% Kolliphor®HS 15 PBS), released rifabutin was analyzed by HPLC (A-D) and compared to freshly prepared RFB in 2% Kolliphor®HS 15 in PBS (E). (F) density of formulations. Release of RFB from implants expressed as daily release rates were evaluated in vitro for 8 weeks (G) F2, (H) F4, n=3, mean and individual replicates are shown.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one skilled in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

As used herein, the terms “extended release” or “long acting” can in some embodiments refer to a substantially sustained release of the drug or active agent over weeks or months, optionally at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 15 weeks, at least about 20 weeks, at least about 30 weeks, or more.

In some embodiments, a “substantially sustained release” of a drug or active agent comprises a release of the drug or active agent from the ISFI at a substantially continuous level over a period of time, e.g. at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 15 weeks, at least about 20 weeks, at least about 30 weeks, or more. In some embodiments, the substantially continuous level of release is within an acceptable degree of variation of the released amount, i.e. within about 1%, 2%, 3%, 4%, 5%, 10%, 15%, 20%, 25%, 30% or 35%.

In some embodiments, an extended release or long acting ISFI as disclosed herein can provide a substantially sustained release of the drug or active agent at a level or concentration sufficient to provide an “effective amount”, a “therapeutically effective amount” or a “Minimal Inhibitory concentration” (MIC) of the drug or active agent. In some embodiments, “effective amount”, a “therapeutically effective amount”, or a “Minimal Inhibitory concentration” refers to an amount of a drug, active agent, compound, composition sufficient to produce a selected effect, such as but not limited to alleviating symptoms of a condition, disease, or disorder. In the context of administering compounds, drugs or active agents in the form of a combination, such as multiple compounds, drugs or active agents, the amount of each compounds, drugs or active agents, when administered in combination with one or more other compounds, drugs or active agents, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

In some embodiments, the effective amount, therapeutically effective amount or MIC is released over weeks or months, optionally at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 15 weeks, at least about 20 weeks, at least about 30 weeks, or more.

### General Considerations of Formulations and In Situ Forming Implants

The disclosure herein demonstrates and shows, for the first time, that changes in the material composition of ISFI formulations can dramatically and unexpectedly increase the drug loading capacity of the ISFI. With increased drug load such an improved ISFI can advantageously provide the ability to release a greater quantity of a desired drug, and/or release the drug or active agent over a longer period of time. Scanning electron microscopy analysis demonstrates that changes in drug load result in structural changes that can be used to modulate pore size and implant erosion resulting in high in vitro and in vivo drug release. By way of example and not limitation, the clinical translational relevance of these observations is demonstrated in the development of a LA rifabutin ISFI formulation (LA-RFB) that 1) delivers high plasma concentration for at least about 16 weeks, 2) prevents acquisition of Mycobacterium tuberculosis (Mtb), and 3) clears acute Mtb infection from the lung and other tissues. These results, as exemplified by LA-RFB, represent a significant advance in material composition with high clinical relevance. It is noted that while rifabutin, in the context of LA-RFB, is used as a proof of concept drug/active agent, other such drugs and active agents suitable for ISFI applications are expected to work in a similar manner in a LA-ISFI.

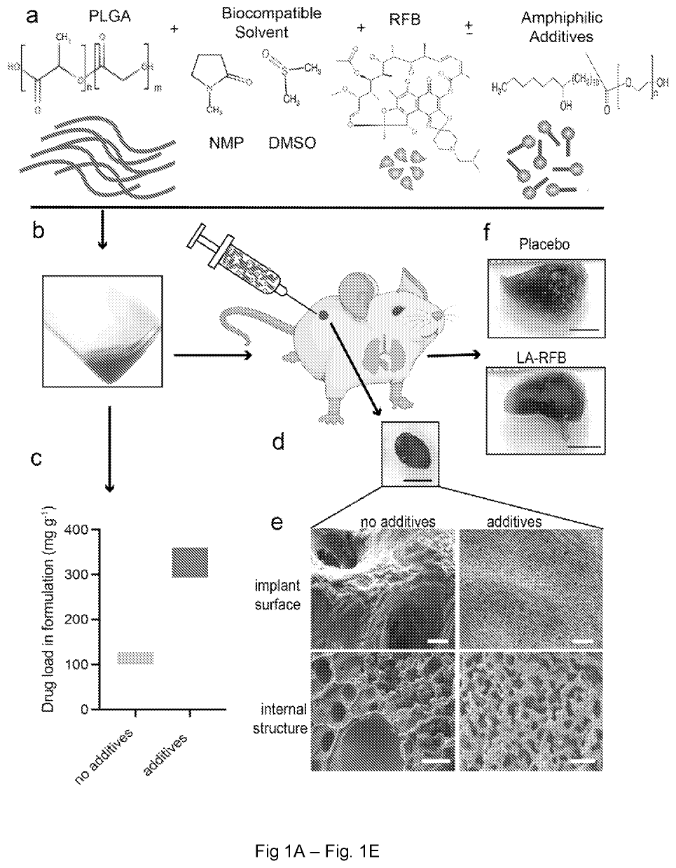

To elaborate, reference is made to FIG. 1, which shows that meaningful changes in the material composition of in situ forming implant formulations result in structural changes, increased payload, reduced erosion, and long-term effective drug delivery. FIG. 1 is a schematic showing LA-RFB (again, RFB is only provided as one exemplary drug/active agent with other drugs/active agents equally suitable for such LA-ISFI applications) composition consisting of, for example, PLGA as the biodegradable polymer, DMSO or NMP as a biocompatible solvent, RFB as an active pharmacological ingredient, and Kolliphor®HS 15 as an example of an additive. FIG. 1B shows that the liquid formulation is injectable and can be administered subcutaneously. As discussed further herein, the range of the drug load increases in LA-RFB formulations after addition of amphiphilic additives (FIG. 1C). FIG. 1D shows a solidified implant of 50 μL LA-RFB, scale bar is 5 mm, with FIG. 1E showing microphotographs of implants without additives (left panel) and with additives (right panel). Scale bar is 2 μm.

Importantly, the present disclosure is the first to demonstrate that amphiphilic additives (surfactants) can surprisingly increase solubility in organic solvent. In previous applications surfactants were always used in some combination with water, not organic solvent only. The unexpected benefit of the increase drug solubility in organic solvent compatible with ISFI formulations using amphiphilic additives including surfactants is and increase drug load in ISFI formulations. The observed increase in drug load was substantial and surprising in a range of about 200% to about 350% or more using the disclosed ISFI formulations. In some embodiment the drug load, i.e. concentration or amount of drug/active agent contained in the composition, is increased by about 200%, about 225%, about 250%, about 275%, about 300%, about 325% or about 350%, or more (or any percentage within the noted range), as compared to an ISFI formulation without the amphiphilic additive.

The disclosed LA-ISFI formulations can be used for any suitable application, with any desired drug/active agent, and for the treatment of any condition or disease where a drug/active agent can be suitably administered from an ISFI. One such application is in the treatment of tuberculosis (TB) and related conditions. Despite global efforts, tuberculosis, which is caused by the bacterium Mycobacterium tuberculosis (Mtb)^{[1, 2]} remains a significant world health concern with high morbidity and mortality.^{[2-4]} According to the World Health Organization (WHO), an estimated 10 million people developed TB in 2020, resulting in 1.5 million deaths.^{[4]} Moreover, approximately one-fourth of the world's population has a latent TB infection (LTBI) with the potential for reactivation.^{[5]} Of additional concern is the rise in drug resistant Mtb.^{[4]} Early diagnosis, treatment of all patients with TB, prevention of LTBI reactivation, and prevention of initial TB infection are all crucial to the WHO strategy in combating the TB epidemic.^{[6]} Additionally, the current COVID-19 pandemic has had a negative impact on TB diagnosis and treatment. It is estimated that the pandemic represents a setback of at least five to eight years in the fight against TB.^{[7]} Prophylaxis with a single anti-TB drug, which acts to prevent initial infection or reactivation of LTBI, is highly effective and can reduce TB incidence when taken consistently.^{[8]} For cases of active drug-susceptible TB disease, multidrug treatment regimens have an intensive phase of two months, followed by a continuation phase of at least four months.^{[9]} Non-adherence to TB treatment can lead to treatment failure and the development of drug resistance, which reduces treatment success. ^{[10.11]} Long acting (LA) parenteral drug formulations that provide sustained drug release over weeks or months, as disclosed herein, have the potential to reduce dosing frequency such that only one or two injections of the drug could be sufficient for TB treatment.^{[12]} This would dramatically change anti-TB treatment, as less frequent dosing would increase treatment compliance and consequently limit the occurrence of drug resistance.^{[6, 13, 14]} Affordable LA anti-TB treatment would also allow the use of this approach in low-income communities where it is most needed.

As disclosed herein, long acting biodegradable formulations based on in situ forming implant (ISFI) technology are attractive due to their unique properties which allow for subcutaneous administration of liquid formulations that solidify and form an implant at the site of injection. Injectable formulations are less invasive and less painful to administer than solid implants, and the biodegradable nature of the polymer matrix eliminates the need for surgical implant removal. However, in the event of serious adverse effects, the implant can be removed and drug delivery stopped. To develop a LA formulation with ISFI properties, the drug of interest and biodegradable polymer can be solubilized in water miscible organic solvents such as dimethyl sulfoxide (DMSO) or N-methyl-2-pyrrolidone (NMP). Upon injection, phase transition occurs by solvent exchange, and polymer precipitation results in the formation of a solid implant consisting of biodegradable polymer and drug. The drug release properties from the implant are controlled by implant structure and polymer biodegradation and can be manipulated by changing the material composition of the liquid formulation. This includes changes in the type of biodegradable polymer, polymer molecular weight, polymer concentration in the formulation, type of solvent, and the presence of additives. As a result, the system can be adapted for a variety of clinical applications and can be formulated for a broad spectrum of drugs.

Although any desired drug or active agent can be used in the disclosed ISFI formulations, one such drug suitable for TB therapy, and used herein to demonstrate proof of concept only and not intended to be limiting, is Rifamycin. Rifamycins, including rifampin (RIF), rifapentine (RFP), and rifabutin (RFB), are the cornerstones of TB therapy due to their potent bactericidal activity and their ability to inhibit DNA-dependent RNA synthesis in prokaryotes.^{[21]} RFB is a hydrophobic drug with reduced potential for drug-drug interactions compared to other rifamycins.^{[22-24]} RFB also has higher tissue uptake, larger volume of distribution, longer terminal half-life, lower minimum inhibitory concentration (MIC) for Mtb, and higher tissue-to-plasma drug concentration ratio compared to RIF.^{[23-27]} In humans, oral administration of 150 mg of RFB resulted in Cₘₐₓ 460 ng mL⁻¹, Cₘᵢₙ 50 ng mL⁻¹, and tissue to plasma ratio of 5.6-6.8 in the lungs.^{[28, 29]} RFB is available as a low-cost generic medication and was selected as a model drug for development of a LA anti-TB drug formulation in this study.

The hybrid nature of ISFI LA formulations (injectable and implant forming) requires a delicate composition balance to accommodate sufficient amounts of drug and polymer solubilized in biocompatible solvent. Currently, this severely limits the amount, number, and types of drugs that can be successfully formulated, and represents a significant challenge when formulating RFB, for example, into an ISFI. As such, the present disclosure provides new LA-ISFI, including a LA-RFB, injectable formulations that in addition to 1) a biodegradable polymer (poly(lactic-co-glycolic-acid) (PLGA), 2) biocompatible water miscible solvent (NMP or DMSO), and 3) active agent, e.g., RFB, also contain 4) an amphiphilic additive that at low concentrations dramatically increased the drug/active agent, including for example RFB, solubility (FIG. 1A, B), 5) It can also contain hydrophobic additives that can increase release of the active agent from the formulation. This results in injectable formulations with high drug load and prolonged stability (FIG. 1C). After injection, these liquid formulations solidify in hydrophilic environments into an implant with small porous microstructures, slow implant erosion and extended drug release (FIG. 1 D,E). In vivo, a single subcutaneous injection of LA-RFB was able to deliver drug for at least four months, efficiently preventing Mtb infection after aerosol exposure, treating established Mtb infection (FIG. 1F), and preventing Mtb dissemination to other organs.

As disclosed herein, these new exemplary LA-ISFI injectable formulations, including LA-RFB, demonstrate a new approach for making and designing LA formulations applicable to numerous active agents, drugs, pharmaceuticals and compounds of interest for any suitable indication or disease.

### Long-Acting Formulations and In Situ Forming Implants for Drug Delivery

The long-acting (LA) formulations, including those for In-Situ Forming Implants (ISFI), and including for example the treatment of Nontuberculous mycobacteria (NTM), can in some embodiments comprise one, some or all the following components.
1. Drug or active agent (see, e.g. Table 1).
2. Biocompatible solvent, including, but not limited to, the following examples:
   Dimethyl sulfoxide (DMSO)
   n-Methyl pyrrolidone (NMP)
   cosolvent system using NMP and DMSO in different DMSO:NMP ratios in the range of 0:100-100:0.
3. Biodegradable polymer, including for example:
   LA formulations (including those with rifabutin and bedaquiline) can be developed with a low molecular weight (MW) polymer, e.g. MW less than about 25 Da, optionally less than about 40 Da, 30 Da, 20 Da, 15 Da or 10 Da.
   Ester capped of acid ending polymer
   Range of lactic acid:glycolic acid ratios of about 50:50 to about 100:0.
4. Amphiphilic additives:
   Table 2 includes an exemplary list of amphiphilic additives that can be use in the system to formulate LA ISFI formulations, including LA-RFB formulations.
5. Hydrophobic additives (optional), including for example:
   Saturated, monounsaturated, and polyunsaturated fatty acids including: Propionic acid, Butyric acid, Valeric acid, Caproic acid, Enanthic acid, Caprylic acid, Pelargonic acid, Capric acid, Undecylic acid, Lauric acid, Tridecylic acid, Myristic acid, Pentadecylic acid, Palmitic acid, Margaric acid, Stearic acid, Nonadecylic acid, Arachidic acid, Heneicosylic acid, Behenic acid, Tricosylic acid, Lignoceric acid, Pentacosylic acid, Cerotic acid, Carboceric acid, Montanic acid, Nonacosylic acid, Melissic acid, Hentriacontylic acid, Lacceroic acid, Psyllic acid, Geddic acid, Ceroplastic acid, Hexatriacontylic acid, Heptatriacontylic acid, Octatriacontylic acid, Nonatriacontylic acid, Tetracontylic acid, linolic acid, eleostearic acid, 9,11-octadecadienoic acid, α-Linolenic acid, Stearidonic acid, Eicosapentaenoic acid, Cervonic acid, Linoleic acid, Linolelaidic acid, γ-Linolenic acid, Dihomo-γ-linolenic acid, Arachidonic acid, Docosatetraenoic acid, Vaccenic acid, Paullinic acid, Oleic acid, Elaidic acid, Gondoic acid, Erucic acid, Nervonic acid, Mead acid.

Fatty alcohols: Hexacosanol, Octacostanol, Dotriacontanol, Butyl alcohol, Amyl alcohol, 3-Methyl-3-pentanol, 1-Heptanol, 1-Octanol, Pelargonic alcohol, 1-Decanol, Undecyl alcohol, Lauryl alcohol, Tridecyl alcohol, Myristyl alcohol, Pentadecyl alcohol, Cetyl alcohol, Palmitoleyl alcohol, Heptadecyl alcohol, Stearyl alcohol, Oleyl alcohol, Nonadecyl alcohol, Arachidyl alcohol, Heneicosyl alcohol, Behenyl alcohol, Erucyl alcohol, Lignoceryl alcohol, Ceryl alcohol, 1-Heptacosanol, Montanyl alcohol, 1-Nonacosanol, Myricyl alcohol, 1-Dotriacontanol, Geddyl alcohol

Terpenes: nerolidol, farnesol.

Sterols: cholesterol, sitosterol, stigmasterol, stigmastanol, ergosterol.

Tocopherols: vitamin E and its derivative.

Regarding the solvent, LA formulations of rifabutin can be developed with DMSO or NMP, or a cosolvent system using NMP and DMSO in different DMSO:NMP ratios in the range of 1:99-99:1. LA formulations of bedaquiline (BDQ) can we developed with NMP or a cosolvent system using NMP and DMSO in different DMSO:NMP ratios in the range of 1:99-50:50

The LA formulations disclosed herein, including those designed for ISFI applications, are suitable for use with numerous drugs and/or active agents depending on the condition or disease to be treated. Table 1 lists exemplary drugs currently used for treatments of TB and NTM infections. They are separated based on effectiveness to specific mycobacterial pathogens. Not all of the drugs in Table 1 are ideally suited for ISFI. At least one criteria for ISFI formulations is low solubility in water, high solubility in biocompatible solvent, and high effectiveness/low therapeutic dose.

Many of the drugs or active agents listed in Table 1 have potential to be developed as long-acting formulations. Drugs notated with a single asterisk (*) are believed to have high solubility in water and thus are unlikely suitable candidates to be formulated in ISFI. However, drugs notated with a double asterisk (**) are those that have a high probability to be formulated using ISFI systems and have similar chemical/physical properties as rifabutin and bedaquiline, for which data already exists (see below). Thus, while any of the drugs in Table 1 are potential candidates, it is expected that ISFI formulations can be most readily developed for any desired drug or active agent, including for those with the similar drug release properties as rifabutin-ISFI and bedaquiline-ISFI formulations.

TABLE 1
Example Drug/Active Agents for LA-ISFI Formulations

| Pathogen | Drug | |
|---|---|---|
| M. tuberculosis | Bedaquiline | ** |
| | Delaminid | |
| | Clofazamine | |
| | Rifapentine | ** |
| | cilastatin | |
| | Moxifloxacin | |
| | Rifabutin | ** |
| | Terizidone | |
| | Prothionamide | |
| | Ethionamide | |
| | Pretamonid | |
| | Rifampin (RIF) | ** |
| | Levofloxacin | |
| | Linezolid | |
| | Capreomycin | |
| | Para-aminosalicylic acid (PAS) | |
| | Ethambutol (EMB) | * |
| | Pyrazinamide (PZA) | * |
| | Imipenem | * |
| | Kanamycin | * |
| | Ioniazid (INH) | * |
| | Amikacin | * |
| | Cycloserine | * |
| | Streptomycin | * |
| | Meropenem (Mpm) | * |
| M. abscessus | Bedaquiline | ** |
| | Clofazamine | |
| | Rifabutin | ** |
| | Cefoxitin | |
| | Clarithromycin | |
| | Tigecycline | |
| | Azithromycin | |
| | Linezolid | |
| | Minocycline | * |
| | Imipenem | * |
| | Apramycin | * |
| | Amikacin | * |
| M. avium complex (MAC) | Rifapentine | ** |
| | Rifabutin | ** |
| | Clarithromycin | |
| | Azithromycin | |
| | Rifampin | ** |
| | Ethambutol (EMB) | * |
| | Amikacin | * |
| M. kansasii | Clarithromycin | |
| | Rifapentine | ** |
| | Rifabutin | ** |
| | Rifampin (RIF) | ** |
| | Ethambutol (EMB) | * |
| | Isoniazid | * |

It should be noted that there are derivatives of bedaquiline under the development with similar structure and properties as bedaquiline that have lower side effects and similar effectiveness as bedaquiline (doi: 10.1128/AAC.02404-19). These drugs/analogs are not listed here, but it is expected that they can be formulated to ISFI and the formulations will have similar release properties as bedaquiline-ISFI.

TABLE 2
Amphiphilic Additives
Name of amphiphilic additives (Non-ionic surfactant)

Kolliphor ®HS 15*
Kolliphor ® RH40
Kolliphor ® EL
Tween 80*
Tween 20*
Vitamin E TPGS*
Polysorbate 40
Polysorbate 60
Poloxamer 124
Poloxamer 188*
Poloxamer 338
Poloxamer 407*
Poloxamer 105
Poloxamer 238
Poloxamer 331
Poloxamer 334
Poloxamer 335
PEG
Span 20
Span 40
Span 80
Span 60
Triton X-100

By way of example only, and not limitation, below are several exemplary LA ISFI formulations with specific drugs/active agents (RFB or BDQ), although other drugs/active agents could be substituted:

### Example 1

RFB: 294 mg mL⁻¹

### Solvent: DMSO

Polymer: 13.5 kDa PLGA (50:50 LA/GA ratio), acid ending
Solvent: polymer ratio: 4:1

### Additive: Kolliphor®HS 15, 0.45% wt

### Example 2

RFB: 294 mg mL⁻¹

### Solvent: DMSO

Polymer: 13.5 kDa PLGA (50:50 LA/GA ratio), acid ending
Solvent: polymer ratio: 4:1

### Additive: TPGS, 1% w/v

### Example 3

RFB: 293 mg mL⁻¹

### Solvent: DMSO

Polymer: 10.6 kDa PLGA (50:50 LA/GA ratio), ester capped
Solvent: polymer ratio: 4:1

### Additive: Kolliphor®HS 15, 0.45% w/v

### Example 4

RFB: 293 mg mL⁻¹

### Solvent: DMSO

Polymer: 10.6 kDa PLGA (50:50 LA/GA ratio), ester capped
Solvent: polymer ratio: 4:1

### Additive: TPGS, 1% w/v

### Example 5

RFB: 352 mg mL⁻¹

### Solvent: NMP

Polymer: 10.6 kDa PLGA (50:50 LA/GA ratio), ester capped
Solvent: polymer ratio: 4:1

### Additive: Kolliphor®HS 15, 0.45% wt

### Example 6

RFB: 352 mg mL⁻¹

### Solvent: NMP

Polymer: 10.6 kDa PLGA (50:50 LA/GA ratio), ester capped
Solvent: polymer ratio: 4:1

### Additive: TPGS 1% w/v

### Example 7

RFB: 352 mg mL⁻¹

### Solvent: NMP

Polymer: 13.5 kDa PLGA (50:50 LA/GA ratio), acid ending
Solvent: polymer ratio: 4:1

### Additive: Kolliphor®HS 15, 0.45% wt

### Example 8

RFB: 352 mg mL⁻¹

### Solvent: NMP

Polymer: 13.5 kDa PLGA (50:50 LA/GA ratio), acid ending
Solvent: polymer ratio: 4:1

### Additive: TPGS, 1% w/v

### Example 9

RFB: 297 mg mL⁻¹

### Solvent: DMSO

Polymer: 10.6 kDa PLGA (50:50 LA/GA ratio), ester-capped
Solvent: polymer ratio: 5:1

### Additive: TPGS, 1% w/v

### Example 10

RFB: 297 mg mL⁻¹

### Solvent: DMSO

Polymer: 13.5 kDa PLGA (50:50 LA/GA ratio), acid-ending
Solvent: polymer ratio: 5:1

### Additive: Kolliphor®HS 15, 0.45% wt

### Example 11

RFB: 293 mg mL⁻¹

### Solvent: DMSO

Polymer: 10.6 kDa PLGA (50:50 LA/GA ratio), ester capped
Solvent: polymer ratio: 2:1

### Additive: Kolliphor®HS 15, 0.45% wt

### Example 12

RFB: 293 mg mL⁻¹

### Solvent: DMSO

Polymer: 10.6 kDa PLGA (50:50 LA/GA ratio), ester capped
Solvent: polymer ratio: 2:1

### Additive: TPGS, 1% w/v

### Example 13

BDQ: 185 mg mL⁻¹

### Solvent: NMP

Polymer: 15 kDa PLGA (50:50 LA/GA ratio), acid ending
Solvent: polymer ratio: 2:1

### Additive: TPGS, 1% w/v

### Example 14

BDQ: 214 mg mL⁻¹

### Solvent: NMP

Polymer: 15 kDa PLGA (50:50 LA/GA ratio), acid ending
Solvent: polymer ratio: 4:1

### Additive: TPGS, 1% w/v

### Multi-Drug Combinations:

The tunability of the ISFI system allows for formulations of multiple drugs (e.g. about 2 to 4 drugs/active agents) in one injection. To illustrate this in an example, rifabutin and bedaquiline were formulated together. In addition, combinations of rifampin-bedaquiline, and rifapentine-bedaquline are expected to be formulated with similar release properties in vitro as the rifabutin-bedaquiline formulation.

Examples of LA-RFB (rifabutin), BDQ (bedaquiline) formulations:

### Example 15

RFB: 325 mg mL⁻¹
BDQ: 140 mg mL⁻¹

### Solvent: DMSO:NMP, 1:1

Polymer: 15.0 kDa PLGA (50:50 LA/GA ratio), acid ending
Solvent: polymer ratio: 4:1

### Additive: TPGS 1%

### Example 16

RFB: 322 mg mL⁻¹
BDQ: 145 mg mL⁻¹

### Solvent: NMP

Polymer: 15.0 kDa PLGA (50:50 LA/GA ratio), acid ending
Solvent: polymer ratio: 4:1

### Additive: TPGS 1%

### Example 17

RFB: 250 mg mL⁻¹
BDQ: 160 mg mL⁻¹

### Solvent: DMSO:NMP, 1:1

Polymer: 15.0 kDa PLGA (50:50 LA/GA ratio), acid ending
Solvent: polymer ratio: 4:1

### Additive: TPGS 1%

### Example 18

RFB: 250 mg mL⁻¹
BDQ: 160 mg mL⁻¹

### Solvent: NMP

Polymer: 15.0 kDa PLGA (50:50 LA/GA ratio), acid ending
Solvent: polymer ratio: 4:1

### Additive: TPGS 1%

Of note, one distinction of the presently disclosed formulations over existing ISFI compositions is that existing formulations do not increase the drug load in the formulation with surfactants. Instead, they are using surfactants to increase solubility in water. This is important for in vitro assay release medium, especially, when the drug is highly insoluble water. In the disclosed formulations surfactant was added into the formulation. It was discovered, and disclosed herein, that amphiphilic additives (surfactants) can increase solubility and drug load in the formulations with organic solvents similarly as observed in water.

In some embodiments, and as exemplified in the working Examples below, one specific formulation of a long-acting injectable that contains four components can be as follows:
1. biodegradable poly(lactic-co5 glycolic-acid) (PLGA, a polymer with molecular weight 10.6 kDa and lactic acid: glycolic acid ratio 50:50);
2. biocompatible water miscible solvent;
3. an amphiphilic additive (Kolliphor HS15, or other additive from Table 2); and
4. active ingredient(s): either rifabutin (RFB), rifapentine, or rifampin, as well as any of the other exemplary drugs and drug combinations disclosed herein, including those for both Nontuberculous Mycobacteria (NTM) and Mycobacterium tuberculosis.

### Preparation of LA ISFI

The Examples section below provides details of the preparation process for the disclosed LA ISFI. Of note, it was surprisingly discovered that the drug load can be increased by changes in the formulation preparation process, including changing the order of steps. Instead of solubilizing the drug in solvent and then adding the polymer, disclosed herein is a method where first the polymer is solubilized in solvent, e.g. DMSO, and then adding the drug/active agent. By way of example and not limitation, a method of making the disclosed ISFIs can comprise solubilizing a biodegradable polymer in a biocompatible solvent containing an amphiphilic additive at a ratio of about 2:1 biocompatible solvent:biodegradable polymer to about 6:1 biocompatible solvent:biodegradable polymer, optionally about 4:1 biocompatible solvent:biodegradable polymer; and adding a drug or active agent to the composition, wherein the drug or active agent is added at a concentration ranging from about 100 mg mL−1 to about 500 mg mL−1, optionally about 200 mg mL−1 to about 400 mg mL−1.

### Example 19

RFB: 400 mg mL⁻¹

### Solvent: DMSO

Polymer: 13.5 kDa PLGA (50:50 LA/GA ratio), acid ending
Solvent: polymer ratio: 4:1

### Additive: Kolliphor®HS 15, 0.45% wt;

### Example 20

RFB: 400 mg mL⁻¹

### Solvent: DMSO

Polymer: 13.5 kDa PLGA (50:50 LA/GA ratio), acid ending
Solvent: polymer ratio: 4:1
Additive: TPGS, 1% w/v;

### Example 21

RFB: 400 mg mL⁻¹

### Solvent: DMSO

Polymer: 10.6 kDa PLGA (50:50 LA/GA ratio), ester capped
Solvent: polymer ratio: 4:1

### Additive: Kolliphor®HS 15, 0.45% wt;

### Example 22

RFB: 400 mg mL⁻¹

### Solvent: DMSO

Polymer: 10.6 kDa PLGA (50:50 LA/GA ratio), ester capped
Solvent: polymer ratio: 4:1
Additive: TPGS, 1% w/v;

### Example 23

RFB: 420 mg mL⁻¹

### Solvent: NMP

Polymer: 10.6 kDa PLGA (50:50 LA/GA ratio), ester capped
Solvent: polymer ratio: 4:1

### Additive: Kolliphor®HS 15, 0.45% wt;

### Example 24

RFB: 420 mg mL⁻¹

### Solvent: NMP

Polymer: 10.6 kDa PLGA (50:50 LA/GA ratio), ester capped
Solvent: polymer ratio: 4:1
Additive: TPGS 1% w/c;

### Example 25

RFB: 420 mg mL⁻¹

### Solvent: NMP

Polymer: 13.5 kDa PLGA (50:50 LA/GA ratio), acid ending
Solvent: polymer ratio: 4:1

### Additive: Kolliphor®HS 15, 0.45% wt;

### Example 26

RFB: 420 mg mL⁻¹

### Solvent: NMP

Polymer: 13.5 kDa PLGA (50:50 LA/GA ratio), acid ending
Solvent: polymer ratio: 4:1
Additive: TPGS, 1% w/v;

### Example 27

RFB: 400 mg mL⁻¹

### Solvent: DMSO

Polymer: 10.6 kDa PLGA (50:50 LA/GA ratio), ester-capped
Solvent: polymer ratio: 5:1
Additive: TPGS, 1% w/v;

### Example 28

RFB: 400 mg mL⁻¹

### Solvent: DMSO

Polymer: 13.5 kDa PLGA (50:50 LA/GA ratio), acid-ending
Solvent: polymer ratio: 5:1

### Additive: Kolliphor®HS 15, 0.45% wt;

### Hydrophobic Additives for Increased Drug Load:

Also disclosed herein is the finding that the release of the drug from the implant can in some embodiments be increased by addition of hydrophobic additives to the formulation. By way of example only, and not limitation, below are several exemplary LA ISFI formulations with specific drugs/active agents (RFB or BDQ), although other drugs/active agents could be substituted, where hydrophobic additives are included to increase drug load:

Examples of RFB-ISFI formulations with hydrophobic additives (Oleic acid as an example):

### Example 29

RFB: 420 mg mL⁻¹

### Solvent: DMSO

Polymer: 13.5 kDa PLGA (50:50 LA/GA ratio), acid ending
Solvent: polymer ratio: 4:1

### Additives: Kolliphor®HS 15, 0.45% wt

Oleic acid, 6.4% w

### Example 30

RFB: 420 mg mL⁻¹

### Solvent: DMSO

Polymer: 13.5 kDa PLGA (50:50 LA/GA ratio), acid ending
Solvent: polymer ratio: 4:1

### Additive: TPGS, 10% w/v

Oleic acid, 6.4% w

### Example 31

RFB: 420 mg mL⁻¹

### Solvent: DMSO

Polymer: 10.6 kDa PLGA (50:50 LA/GA ratio), ester capped
Solvent: polymer ratio: 4:1

### Additive: Kolliphor®HS 15, 0.45% wt

Oleic acid, 6.4% wt

### Example 32

RFB: 420 mg mL⁻¹

### Solvent: DMSO

Polymer: 10.6 kDa PLGA (50:50 LA/GA ratio), ester capped
Solvent: polymer ratio: 4:1

### Additive: TPGS, 10% w/v

Oleic acid, 6.4% wt

### Example 33

RFB: 420 mg mL⁻¹

### Solvent: DMSO

Polymer: 13.5 kDa PLGA (50:50 LA/GA ratio), acid ending
Solvent: polymer ratio: 4:1

### Additives: Kolliphor®HS 15, 0.45% wt

Oleic acid, 12% wt

### Example 34

RFB: 420 mg mL⁻¹

### Solvent: DMSO

Polymer: 13.5 kDa PLGA (50:50 LA/GA ratio), acid ending
Solvent: polymer ratio: 4:1

### Additive: TPGS, 10% w/v

Oleic acid, 12% wt

### Example 35

RFB: 420 mg mL⁻¹

### Solvent: DMSO

Polymer: 10.6 kDa PLGA (50:50 LA/GA ratio), ester capped
Solvent: polymer ratio: 4:1

### Additive: Kolliphor®HS 15, 0.45% wt

Oleic acid, 12% wt

### Example 36

RFB: 420 mg mL⁻¹

### Solvent: DMSO

Polymer: 10.6 kDa PLGA (50:50 LA/GA ratio), ester capped
Solvent: polymer ratio: 4:1

### Additive: TPGS, 10% w/v

Oleic acid, 12% wt

### Example 37

RFB: 420 mg mL⁻¹

### Solvent: DMSO

Polymer: 13.5 kDa PLGA (50:50 LA/GA ratio), acid ending
Solvent: polymer ratio: 4:1

### Additives: Kolliphor®HS 15, 0.45% wt

Oleic acid, 21.4% wt

### Example 38

RFB: 420 mg mL⁻¹

### Solvent: DMSO

Polymer: 13.5 kDa PLGA (50:50 LA/GA ratio), acid ending
Solvent: polymer ratio: 4:1

### Additive: TPGS, 10% w/v

Oleic acid, 21.4% wt

### Example 39

RFB: 420 mg mL⁻¹

### Solvent: DMSO

Polymer: 10.6 kDa PLGA (50:50 LA/GA ratio), ester capped
Solvent: polymer ratio: 4:1

### Additive: Kolliphor®HS 15, 0.45% wt

Oleic acid, 21.4% wt

### Example 40

RFB: 420 mg mL⁻¹

### Solvent: DMSO

Polymer: 10.6 kDa PLGA (50:50 LA/GA ratio), ester capped
Solvent: polymer ratio: 4:1

### Additive: TPGS, 10% w/v

Oleic acid, 21.4% wt

The LA ISFI formulations, or extended release or long-acting injectable compositions, disclosed herein, particularly those with the hydrophobic additive, saw increased release of the drug or active agent from a solidified implant formed from the extended release or long-acting injectable composition by at least 5%, optionally by about 20% to about 150%, as compared to a solidified implant without a hydrophobic additive. In some embodiments, such LA ISFIs with one or more hydrophobic additives showed improved drug release of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150% or more.

Moreover, it was surprisingly disclosed that autoclaving of the ISFI-LA formulations is a suitable method of terminal sterilization without changes in formulations critical quality attributes (formulation density, injectability, implant formation and in vitro release properties).

### Applications and Indications:

The disclosed LA ISFI compositions and formulations can be used to treat numerous conditions and diseases in subjects in need of treatment, and can be modified with an appropriate drug and/or active agent as needed depending on the condition or disease to be treated. Below includes further discussion of some example conditions to be treated.

Mycobacterium tuberculosis, the bacterium responsible for tuberculosis (TB), is a major global health problem. Currently, M. tuberculosis ranks second only to SARS CoV-2 as a leading cause of death by an infectious agent. Moreover, the COVID-19 pandemic has set back efforts to control TB with more TB deaths reported in 2020 than in past decades, which is attributed to delayed TB diagnosis and treatment disruptions. Of the 1.5 million TB deaths in 2020, 214,000 deaths were in HIV infected individuals making M. tuberculosis the leading cause of death of people living with HIV. However, while immunocompromised HIV+ people are at higher risk for developing TB, the majority of TB cases and deaths are not in HIV co-infected people. The primary manifestation of TB is chronic pulmonary disease (chronic pneumonia), but TB can also cause disseminated disease (miliary TB) and extrapulmonary disease (e.g. lymph nodes, bone, abdomen etc.).

Treatment for active TB: For drug susceptible strains, four first line drugs are given for 6-9 months. The standard regimen is as follows: Rifampin (Rifapentine or Rifabutin as alternatives), Isoniazid, Pyrazinamide, Ethambutol.

Treatment with this first line therapy is for 2 months (intensive phase) followed by rifampin and isoniazid for 4 months (continuation phase). For HIV+ patients, there is concern about rifampin interacting with antiretrovirals and reverse transcriptase inhibitors used to treat HIV.

Rifabutin is another member of the rifamycin drug family that is effective on M. tuberculosis. Rifabutin has fewer drug interactions and is an alternative to rifampicin for treating TB.

Treatment for latent TB is either isoniazid and/or rifampin or rifapentine for 3-9 months. Alternatively, rifampin alone may be used.

Treatment for drug resistant TB: Multidrug-resistant (MDR) TB is caused by M. tuberculosis that is resistant to at least isoniazid and rifampin (RIF). Extensively drug-resistant (XDR) TB is an MDR TB case that is additionally resistant to any fluoroquinolone and at least one of three injectable second-line drugs (i.e., amikacin, kanamycin, or capreomycin). Second and third line drugs to treat MDR TB and XDR TB are as follows (note that not all drugs in the list are suitable for development of LA formulations and not all of them could increase solubility in presence of amphiphilic additives, additional drug list that identifies drugs suitable for LA formulation (see also Table 1): Bedaquiline, Linezolid, Pretarnonid, Moxifloxacin, Levofloxacin, Para-aminosolcylic acid, Delaminid, Capreomycin, Kanamycin, Anikacin, Streptomycin, Cycloserine, Prothionamide, Rifapentine, Rifabutin, Clofazimine, Tenzidone, Ethionarnide, Imipenern-cilastatin, Meropenem.

Treatment for MDR and XDR-TB has been multidrug therapy with different combinations of the above drugs for 2 years or greater, with a 50% success rate. A recent breakthrough for XDR TB treatment is the BPaL regimen (comprised of bedaquiline, pretomanid, and linezolid), which significantly shortens the time of treatment of XDR-TB from ≥2 years to 6-9 months.

Nontuberculous mycobacteria(NTM) are a large group of environmental my cobactenia, some of which are important causes of human disease. Chronic pulmonary disease is the most common manifestation of NTM infection, and it associated with morbidity and mortality. However, NTMs can also cause skin and soft tissues infections and disseminated disease. Patients at the highest risk for NTM pulmonary disease are those with chronic obstructive pulmonary disease (COPD), alpha-1-antitrypsin (AAT) deficiency, cystic fibrosis (CF), non-CF bronchiectasis, primary ciliary dyskinesia, silicosis, emphysema, Sjorgen's syndrome, or immune suppression due to primary immune deficiency syndromes such as Mendelian Susceptibility to Mycobacterial Disease (MSMD). Individuals who do not have identifiable risk factors can also get pulmonary disease and there is a poorly understood association between NTM disease and older women with a slender body habitus and thoracic cage abnormalities, such as scoliosis. The most common species associated with NTM disease are in the Mycobacterium avium complex (MAC), which can be associated with HIV infection, or subspecies of Mycobacterium abscessus. There are three subspecies of M. abscessus: M abscessus subsp. massiliense, M abscessus subsp. bolletii and M abscessus subsp. abscessus. M. kansasii is another NTM associated with chronic pulmonary disease. Of the NTM pathogens, M. abscessus species are the most difficult to treat due intrinsic multidrug resistance. In the United States, it is estimated that 86,000 people or greater are living with NTM pulmonary disease. Studies from around the world indicate increasing incidence and prevalence of NTM pulmonary disease in the US and elsewhere.

Treatment for NTM infection

M. abscessus treatment. M abscessus is the hardest NTM to treat. There is no systematically proven regimen to treat M. abscessus (doi: 10.1136/thoraxjnl-2015-207983; doi: 10.1136/bmjresp-2017-000242) and the term “incurable nightmare” is often used to describe it. Several years of treatment with a minimum of three antibiotics is not uncommon for M abscessus and the cure rate is only 30%-50% with disease relapse a common occurrence. In some cases of localized disease, surgical lung resection is advised. Drugs used to treat M. abscessus are: Clofazamine, Cefoxitin, Tigeclyin, Azithromycin, Clarithromycin, Linezolid, Mincoycline, Imipenem, Aparamycin, Amikacin. Rifabutin is in development for treating M abscessus in human patients and there is a recent case report of it being used to treat M. abscessus infection. Note rifampin and rifapentine, which are effective in treating M. tuberculosis, do not work on M abscessus. Bedaquiline is in development for treating M abscessus in human patients and there are couple of case reports for it being used as a salvage therapy. A very recent paper discusses the antibacterial efficacy of combination rifabutin and bedaquiline on in vitro growing M abscessus. When combined, these drugs were able elicit bactericidal activity at relatively low, clinically relevant concentrations. This is significant as both these drugs are compatible with the long-acting formulations in the subject disclosure.

M. avium treatment: Rifampin, Rifabutin, Rifapentine, Azithromycin, Clarithromycin, Streptomycin, Amikacin, Ethambutol.

M. kansasii treatment: Rifampin, Rifabutin, Rifapentine, Ethambutol, Isoniazid, Clarithromycin.

Advantages of Long-Acting (e.g. Monthly) Subcutaneous Injections of Drugs

The treatment of M. tuberculosis or NTM disease is always multidrug therapy. For drug resistant disease it is not uncommon for patients to be on daily >4 drug therapy. Some of the drugs used are administered by IV injection (example IV amikacin). Moreover, NTM disease is a growing problem for patients with underlying lung diseases, such as CF, and these patients are taking many additional medications to manage their CF disease and other associated infections. The length and complexity of M. tuberculosis or NTM disease treatment, together with treatment of preexisting conditions often leads to low adherence to medications. Nonadherence to treatment regimens can lead to treatment failure and the development of drug resistance. One way to potentially enhance patient compliance with treatment regimens is the use of long acting (LA) parenteral drug formulations that provide sustained drug release over weeks or months. LA formulations will reduce the frequency of dosing, can reduce the incidence of new TB/NTM infections, and can limit the occurrence of drug resistance. As disclosed herein, LA formulations were developed for M. tuberculosis or NTM based on in situ forming implant (ISFI) technology. ISFIs are injectable drug formulations that solidify after administration. They are less invasive and less painful to administer than solid implants, and the biodegradable nature of the polymer matrix eliminates the need for surgical implant removal. However, in the event of serious adverse effects, the implant can be removed, and drug delivery stopped. Thus, a long-acting subcutaneous formulation of one of the many drugs in the regimen or of multiple drugs in the regimen would have the advantage of convenience and increased patient compliance.

### Subjects

The subject(s) treated with the disclosed LA and/or LA ISFI formulations and compositions are desirably a human subject, although it is to be understood that the principles of the disclosed subject matter indicate that the compositions and methods are effective with respect to invertebrate and to all vertebrate species, including mammals, which are intended to be included in the term “subject”. Moreover, a mammal is understood to include any mammalian species in which screening is desirable, particularly agricultural and domestic mammalian species.

The disclosed methods are particularly useful in the treatment of warm-blooded vertebrates. Thus, the presently disclosed subject matter concerns mammals and birds.

More particularly, provided herein is the treatment of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economic importance to humans. Thus, provided herein is the treatment of livestock, including, but not limited to, domesticated swine (pigs and hogs), ruminants, horses, poultry, and the like.

In some embodiments, the subject to be used in accordance with the presently disclosed subject matter is a subject in need of treatment. In some embodiments, a subject can have or be believed to have a TB, NTM or related disease, condition or phenotype.

### Uses and Pharmaceutical Compositions

In some embodiments, provided are uses of a therapy for a disease, condition or phenotype in a subject, whereby the disease, condition or phenotype in the subject is treated. The therapies in such uses include the same methods and compositions for treating diseases, conditions or phenotypes as disclosed herein, particularly the disclosed LA ISFI compositions. The compositions can be for use in the preparation of a medicament for treating a disease, condition or phenotype.

In some embodiments, provided are uses of a therapy for TB, NTM or related disease, condition or phenotype in a subject, whereby TB, NTM or related disease, condition or phenotype in the subject is treated. The therapies in such uses include the same methods and compositions for treating TB, NTM or related disease, condition or phenotype as disclosed herein, particularly the disclosed ISFI compositions. The compositions can be for use in the preparation of a medicament for treating TB, NTM or related disease, condition or phenotype.

In some embodiments, the presently disclosed subject matter provides a pharmaceutical composition, which can include a pharmaceutically acceptable carrier. In some embodiments, the compounds of the presently disclosed subject matter are formulated for use in treating TB, NTM or related disease, condition or phenotype, or any other disease or condition. The compositions can be prepared for subcutaneous, parenteral, or other administration, such as using a formulation known in the art for preparing an agent/drug/active agent for treating another indication known to be treated by the agent.

In some embodiments, compositions of the presently disclosed subject matter are provided for use in the treatments as disclosed herein, such as for use in the treatments in humans and in animals. In some embodiments, compositions can be provided for use in combination with each other.

In some embodiments, the method further comprises administering one or more additional therapeutic agents to the animal subject. The one or more additional therapeutic agents can be an agent use to treat or mitigate one or more symptoms in the subject. By way of example and not limitation, the additional therapeutic agent can be a therapeutic agent for treating fever or pain.

In some embodiments, the compositions of the presently disclosed subject matter can be provided as pharmaceutical compositions and be provided in pharmaceutically acceptable carriers. In some embodiments, the compositions can be provided as a pharmaceutically acceptable salt. Such salts include, but are not limited to, pharmaceutically acceptable acid addition salts, pharmaceutically acceptable base addition salts, pharmaceutically acceptable metal salts, ammonium and alkylated ammonium salts, and combinations thereof.

Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like.

Base addition salts include but are not limited to, ethylenediamine, N-methyl-glucamine, lysine, arginine, ornithine, choline, N, N′-dibenzylethylenediamine, chloroprocaine, diethanolamine, procaine, N-benzylphenethylamine, diethylamine, piperazine, tris (hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like.

Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

In some embodiments, the presently disclosed compounds can further be provided as a solvate.

In some embodiments, carriers suitable for use in the presently disclosed subject matter include, but are not limited to, alcohols (including benzyl alcohol and its derivatives, monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration.

Solid carriers suitable for use in the presently disclosed subject matter include, but are not limited to, inert substances such as lactose, starch, glucose, methylcellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Materials, Methods and Experimental Designs for Examples 1-11

### Materials

Poly (D,L-lactic-co-glycolic acid) (Lactic acid (LA):glycolic acid (GA)=50:50, ester end group, (PLGA)) polymers with different molecular weights (MW: 36.8, 22.9, or 10.6 kDa) were purchased from LACTEL® Absorbable Polymers (AL, USA). Rifabutin (RFB) and rifampin (RIF) were purchased from Cayman Chemical (MI, USA). Dimethyl sulfoxide (DMSO), acetonitrile with 0.1% formic acid, and Tween 80 were bought from Fisher Scientific (MA, USA). N-Methyl-2-pyrrolidone (NMP), ethylenediaminetetraacetic acid (EDTA), Kolliphor®HS 15 (Solutol), pluronic F127, F68, Tween 20, D-α-Tocopherol polyethylene glycol 1000 succinate (TPGS), phosphate-buffered saline (PBS, pH 7.4), and PLGA with acid-ending (LA:GA=50:50, acid end group, Resomer® RG 502 H, 13.5 kDa) were purchased from Sigma-Aldrich (MO, USA).

### RFB Solubility in DMSO and NMP

DMSO or NMP (50 L) was added to 15 mg of RFB. The mixed samples were incubated for 1 day at room temperature (RT) with 8 rpm using a rotary shaker (Barnstead/Thermolyne Model 415110 Labquake Shaker/rotator, Thermo Fisher Scientific, USA). The undissolved RFB in the solvent was removed by centrifugation (15000 rpm, RT, 5 min) (Eppendorf Centrifuge 5417R, Eppendorf Inc., Germany) and RFB concentration in the supernatant was analyzed by measuring the absorbance at 320 nm using a UV-vis spectrometer (SpectraMax M2/M2e microplate reader, Molecular devices, CA, USA and DeNovix DS-11, Wilmington, DE, USA). All experiments were performed in triplicate.

RFB Solubility with Different Amphiphilic Additives

The effect of different amphiphilic additives on RFB solubility was investigated using various concentrations in biocompatible solvents. Kolliphor®HS 15 was prepared in DMSO (concentration range: 0.045-47.6 wt %) and NMP (concentration range: 0.45-47.6 wt %). Pluronic F127, F68, Tween 80, Tween 20, and TPGS were dissolved in DMSO at concentration range: 0.0009-8.3 wt %. RFB was then mixed with the prepared solutions to a target of 900 mg mL⁻¹ RFB solution, and the mixtures were incubated on a rotary shaker for 1 day at RT. The undissolved RFB in the solution was removed by centrifugation (15000 rpm, 5 min, RT), and the supernatant was collected. RFB concentration in the supernatant was measured by absorbance using a UV-vis spectrometer (DeNovix DS-11). All experiments were performed in triplicate.

### Preparation of LA-RFB Formulations

RFB was mixed with the solvent (DMSO or NMP) with or without 0.45 wt % Kolliphor®HS 15 at the maximum saturated concentration and incubated in a rotary shaker for 1 day at RT. PLGA at different mass ratios to solvent (solvent: polymer=2:1, 4:1 or 5:1) was added and incubated in the rotary shaker over 1 day at RT in the dark or until polymer dissolved. RFB9Sol=9 and RFB14Sol=14 formulations were prepared by dissolving RFB in DMSO with 0.45 wt % Kolliphor®HS 15 at concentration 202 mg mL⁻¹. PLGA ester-capped (RFB9Sol=9) or acid-ending (RFB14Sol=14) was added at solvent: polymer=4:1 mass ratio and mixture was incubated in rotary shaker for 1 day to dissolve polymer. The prepared formulations were then stored in the dark at RT. Drug load (%) was calculated according to equation 1:^{[30]}

$$Drug load ( \% ) = \frac{weight of drug}{weight of ( drug + polymer )} \times 100 \qquad ( 1 )$$

### In Vitro Release Properties of LA-RFB Formulations

RFB in vitro release from LA-RFB was evaluated in phosphate buffered saline (PBS, pH 7.4), at 37° C. in an orbital shaker (100 rpm) under sink conditions (concentration of RFB in release medium <42 μg). Specifically, 30 μl of the prepared RFB-ISFI formulation was directly injected into 10 mL of PBS (pH 7.4) using a 1 mL syringe with a 19-gauge needle. At predetermined time points (1, 2, 3, 5, 7, 10, 14 days, and once a week up to 14 weeks of incubation), 1 mL of solution was collected, and the total release buffer was replaced with fresh PBS. After 14 weeks, the remaining implants were collected and dissolved in 1 mL DMSO. The RFB concentration in the collected release medium and the dissolved implant samples were measured using a UV-vis spectrometer. All experiments were performed in triplicate. RFB release was evaluated as cumulative release of RFB in g or percentage of injected RFB^{[31, 2]} (equation 2). The release rate of RFB^{[33]} at each sampling time point were calculated according to the equation 3:

$$Cumulative RFB percentage ( \% ) = \frac{{V}_{0} {\sum }_{i = 1}^{n} {C}_{i}}{{m}_{0}} \times 100 \qquad ( 2 )$$

$$RFB release rate ( µg {day}^{- 1} ) = \frac{{V}_{0} ( {C}_{n} - {C}_{n - 1} )}{{t}_{n} - {t}_{n - 1}} \qquad ( 3 )$$

Where V₀ is the total volume of release media, Cᵢ is the RFB concentration (mg mL⁻¹) in the release solution, m₀ is the weight of RFB in the formulations injected to release medium, and to is time (days) of sample collected, Cₙ is the RFB concentration in release media at day of collection. Initial release burst was defined as cumulative percentage of total RFB released within the first 72 hours of incubation in release medium.

### In Vivo Pharmacokinetics of LA-RFB Formulations

All animal protocols were approved by the Institutional Use and Care Committee at the University of North Carolina-Chapel Hill and with reference to the National Institutes of Health Guide for the Care and Use of Laboratory Animals, approval number 21-134. BALB/c mice were administered subcutaneously with 50 μL of the LA-RFB formulation using a 19-gauge needle. At predetermined time points (3, 7, 14, 21, 28, 35, and 42, then biweekly until plasma RFB concentrations decreased below the MIC. Peripheral blood was collected in EDTA coated tubes and plasma isolated by centrifugation (5 min, 300 g) and stored at −80° C. until analysis by HPLC.

### HPLC Analysis of Plasma Rifabutin Concentrations

Plasma RFB concentrations were measured by HPLC analysis using rifampin (RIF) as an internal standard.^{[34]} Briefly, 36 μl of plasma was mixed with 4 μl of 100 μg mL⁻¹ RIF solution in DMSO followed by the addition of 60 μL of acetonitrile with 0.1% formic acid. Excess protein was removed by centrifugation (5 min, 12000 rpm, 4° C.) (Eppendorf 5424 Microcentrifuges, Eppendorf Inc., Germany). Supernatant (80 μl) was then collected and dried for 1 h using a vacuum centrifuge (Eppendorf 5301 Vacufuge Concentrator Centrifuge, Eppendorf Inc., Germany). The dried sample was resuspended in 32 μL of acetonitrile with 0.1% formic acid. The resuspended samples were centrifuged (5 min, 12000 rpm, 4° C.) and supernatants collected. RFB concentration in supernatant was measured using HPLC (Agilent Technologies 1200 series HPLC system, Agilent Scientific Instruments, USA). HPLC condition: 40° C., running time=10 min, C18 column with a mobile phase composed of cosolvent (acetonitrile:water=60:40) at a rate of 1 mL min⁻¹; detection wavelength: 275 nm).

### Analysis of RFB in Tissues

RFB in mouse tissue was extracted from calibration standards, quality control samples, and study samples using protein precipitation and LC-MS/MS analysis. Tissue samples were initially homogenized in phosphate buffered saline (PBS) with 100 μg mL⁻¹ cycloheximide and 50 μg mL⁻¹ carbenicillin. The resulting homogenate was mixed with methanol in a 1:3 ratio. Fifty μL of resulting sample was extracted by protein precipitation with methanol containing RFB-d₇ (RFB-IS) as an internal standard. Following vortex and centrifugation, a portion of the supernatant was diluted with water 1:1 prior to LC-MS/MS analysis. RFB was eluted from a Phenomenex Synergi Polar-RP (50×2.0 mm, 2.5 um particle size) analytical column. Data were collected using Sciex Analyst Chromatography Software on an API-5000 triple quadruple mass spectrometer (SCIEX, Foster City, CA, USA). Calibration curves were obtained by using a 1 concentration⁻² weighted linear regression of analyte:internal standard peak area ration vs. concentration. The calibration curve for this assay was 1-20,000 ng mL⁻¹ homogenate. All calibrators and quality control samples were within 15% of the nominal concentrations.

### Stability of LA-RFB Formulations

RFB14 and RFB14Sol were stored at room temperature in the dark. At predetermined time points (3, 4, 6, 9, 12, 15 months of incubation) 10 μL of formulation was diluted in 1 mL of DMSO, then further diluted at 1:1000 in DMSO for analysis by UV-vis absorbance spectrometer and HPLC to measure the drug concentration and drug degradation. As a control, RFB dissolved in DMSO (20 μg mL⁻¹ RFB solution) was incubated in the dark at RT.

### Scanning Electron Microscopy (SEM) Imaging of LA-RFB Implants

Implants were prepared by injection of 30 μL LA-RFB formulation into 10 mL of PBS (pH 7.4) and incubated at 37° C. with 100 rpm shaking. The PBS buffer was replaced with fresh PBS following the same time schedule as the in vitro release experiment. At pre-determined time points (3, 7, and 28 days after incubation), the implants were collected and lyophilized for 24 h. The lyophilized implants were fractured by razor on dry ice to investigate their internal structure. Implants were mounted on an aluminum platform using carbon tape. The mounted implants were coated with 5 nm of gold-palladium alloy (60:40) (Hummer X Sputter Coater, Anatech USA, Union City, CA). The coated samples were imaged using a Zeiss Supra 25 field emission scanning electron microscope with an acceleration voltage of 5 kV, 30 m aperture, and average working distance of 10 mm (Carl Zeiss Microscopy, LLC, Thornwood, NY).^{[17, 35]} Pore size and porous area on the surface of implants were measured from the SEM images using Image J software (NIH, Maryland).^{[36]} The porous area was calculated as a percentage of pore area in total area of the scanned SEM image. The pore size and porous area on the surface implants were measured on three regions of each implant, one to three different implants were analyzed.

### Mtb Infection and LA-RFB Efficacy In Vivo

All studies involving Mtb were carried out under Biosafety Level 3 (BSL-3) containment. BALB/c mice were transferred to the BSL-3 facility and allowed to rest for one week prior to experimentation.

Mtb Erdman was grown at 37° C. in liquid Middlebrook 7H9 medium supplemented with 0.05% Tween 80, 0.5% glycerol and 1× albumin-dextrose-saline (>0.5% bovine serum albumin, 0.2% glucose, 0.85% NaCl). Mice were placed in a Madison aerosol chamber (Mechanical Engineering Workshop, Madison, WI) calibrated to deliver ˜250 CFU Mtb. After each exposure, four mice were sacrificed one day post infection to determine bacterial uptake. Whole lungs were weighed and then homogenized in PBS supplemented with 100 μg mL⁻¹ cycloheximide, and 50 μg mL⁻¹ carbenicillin. Diluted homogenate was then plated on 7H10 plates supplemented with 0.5% glycerol, 10% OADC (oleic acid, bovine albumin, dextrose, and catalase), 100 μg mL⁻¹ cycloheximide, 50 μg mL⁻¹ carbenicillin, and 15 μg mL⁻¹ trimethoprim for CFU enumeration.

Either two weeks prior to exposure or one week after exposure, mice received a single subcutaneous injection of 50 μL LA-RFB or placebo using a 1 mL syringe and 19-gauge needle. Groups of mice were sacrificed at 28 days post-infection, and lungs, livers, and spleens collected for analysis. A small piece of tissue was retained for histology in 10% formalin over 24 h prior to removal from the BSL-3. The remaining tissue was weighed and processed as with whole lungs for CFU enumeration.

Fixed tissue was removed from the BSL-3, paraffin embedded, and cut into 5 m sections, which were mounted onto Superfrost Plus slides (Fisher Scientific). Tissue sections were incubated at 60° C. for one hour, followed by deparaffinization with xylene (2×3 min) and rehydration in graded ethanol (100% 2×3 min, 95% 1×3 min, 80% 1×3 min, 70% 1×3 min) and ddH₂O (10 min). Sections were stained with hematoxylin for 15 min, washed in water for 20 min, and stained with eosin for one min. Following ethanol and xylene incubation (95% ethanol 2×2 min, xylene 2×2 min), slides were mounted and imaged on a Nikon Eclipse Ci microscope using Nikon Elements BR software (version 4.30.01) with a Nikon Digital Sight DS-Fi camera. ImageJ/Fiji (version 2.1.0/1.53f) was used to adjust brightness and contrast on whole images.

### Statistical Analysis

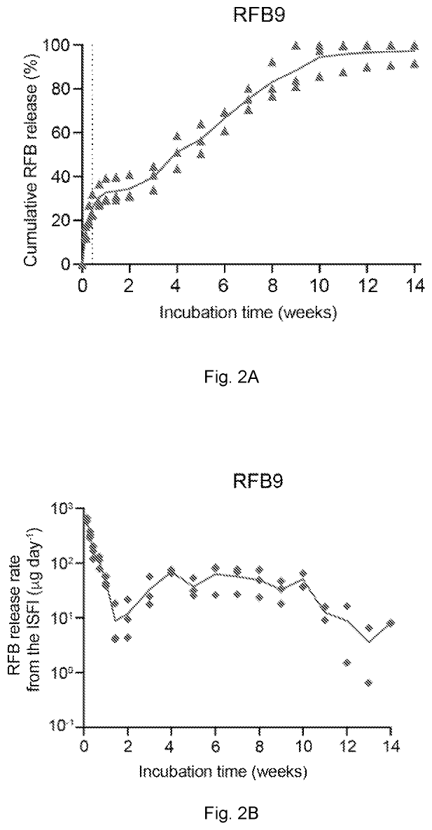

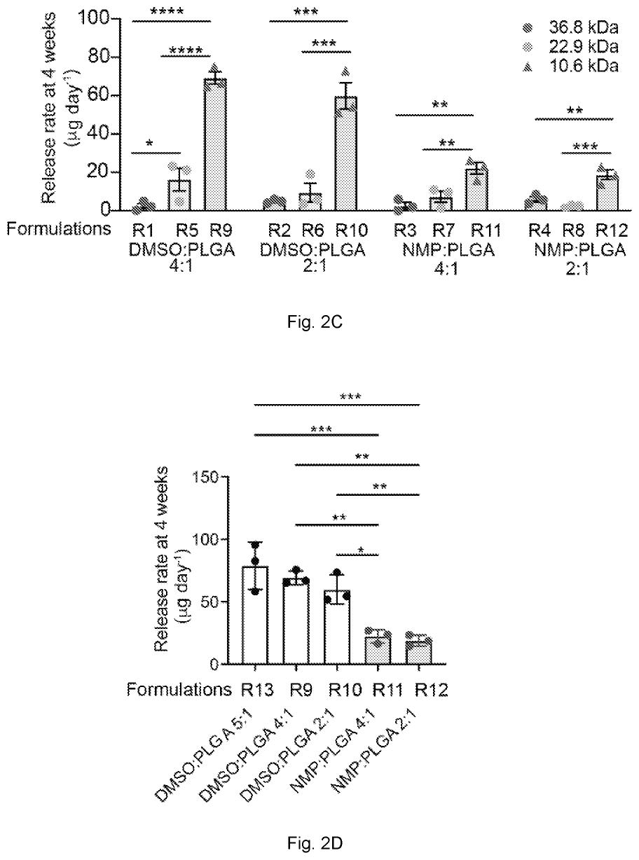

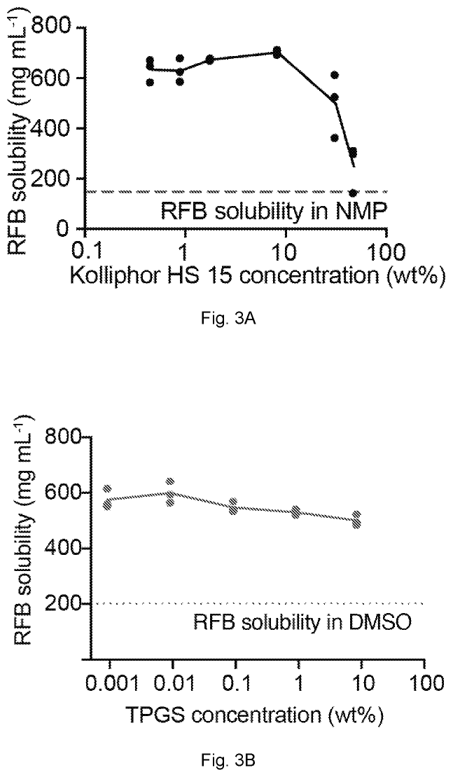

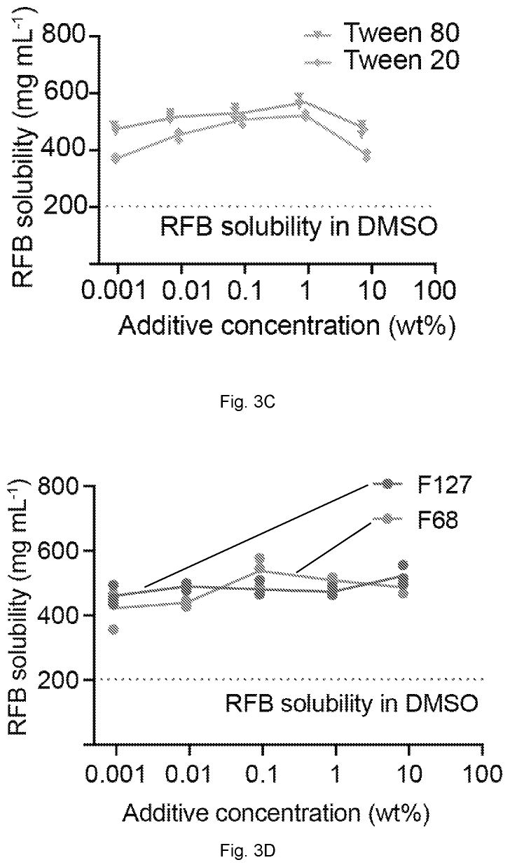

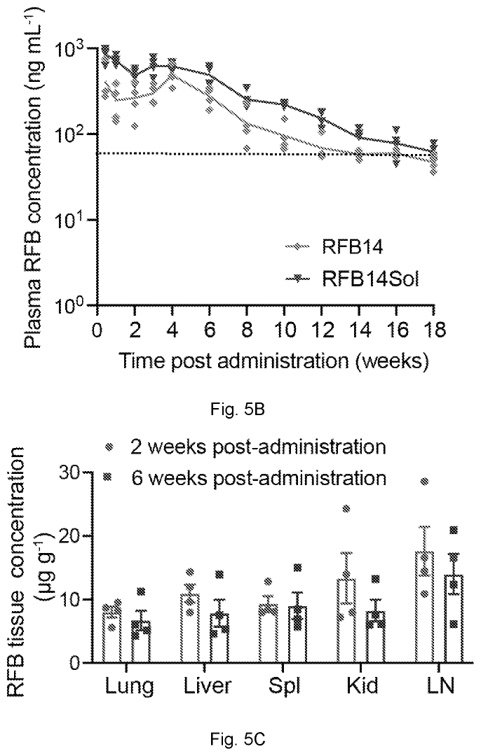

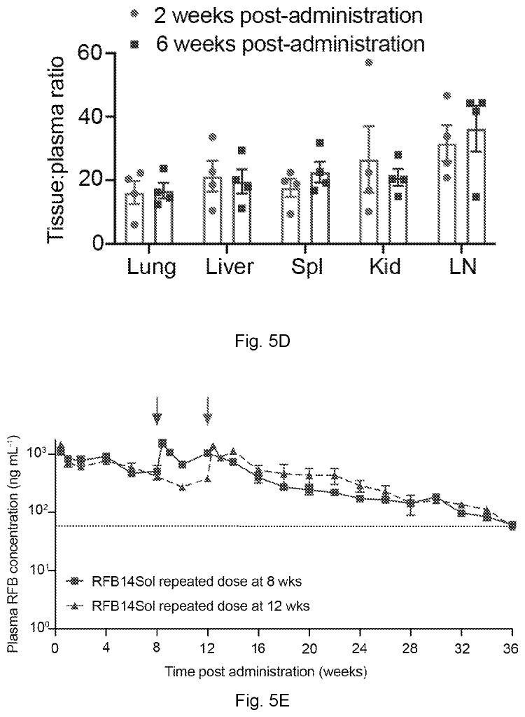

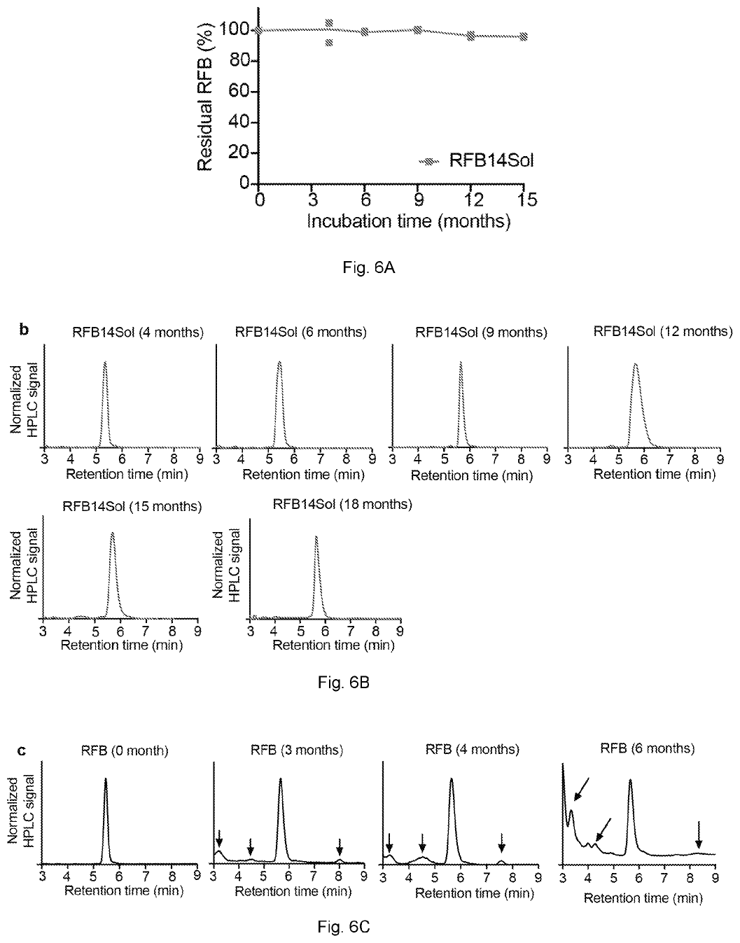

No statistical methods were used to predetermine sample size. All statistical analyses were completed using GraphPad Prism (version 9.0.0). Differences in release rates from LA-RFB formulations based on polymer MW were determined using a one-way ANOVA for each solvent to polymer ratio (Supplemental FIG. 1c). Correction for multiple comparisons was controlled using the False Discovery Rate and the two-stage step-up method of Benjamini, Krieger, and Yekutieli. Similarly, a one-way ANOVA followed by correction for multiple comparisons was used to compare all formulations with a low MW PLGA (Supplemental FIG. 1d). Differences in RFB drug load and initial release burst were compared using an unpaired t-test (FIG. 2b,d) Differences in the RFB tissue concentration and the tissue to plasma ratio at two and six weeks post treatment were analyzed using a Mann-Whitney U test for each organ (FIG. 3c,d). Differences in the porous area and pore size for each implant over time (FIG. 5b,c) were analyzed using a one-way ANOVA for each formulation with multiple comparisons as in Supplemental FIG. 1c. To determine the difference between CFU burden in treated and untreated animals (FIG. 6b,g), a Mann-Whitney U test was completed for each organ. For all figures, significance is represented by asterisks as follows *: 0.01<p≤0.05; **: 0.001<p≤0.01; ***: 0.0001<p≤0.001; and ****: p≤0.0001.

### Example 1

### Amphiphilic Additives Enhance RFB Solubility and Improve LA-RFB Release Kinetics

To achieve the highest possible drug release from the solidified implants, the polymer and solvent composition of LA-RFB formulations was manipulated. LA-RFB formulations were prepared by dissolving RFB at the maximum solubility in biocompatible solvents DMSO or NMP (202±22 and 148±34 mg mL⁻¹, respectively, mean±SD). Biodegradable ester-capped PLGAE^{[35]} polymer with different molecular weights (MW, 10.6 kDa, 22.9 kDa, or 36.8 kDa and an LA:GA ratio of 50:50) was added to the RFB solution at multiple solvent to polymer mass ratios (2:1, 4:1, and 5:1) (Table 3). The resulting formulations were evaluated for 1) their injectability using a 1-mL syringe with a 19-gauge needle, 2) implant formation after injection to aqueous release medium (PBS), 3) initial release burst, defined as a release of RFB (%) within the first 72 h of incubation in release medium, and 4) daily release rate (g day⁻¹) at 37° C. with 100 rpm shaking under sink conditions (concentration of RFB in release medium <42 μg mL⁻¹).

TABLE 3
In vitro characteristics of LA-RFB formulations

| | | | | | | Initial | Release rate | Release rate | Release rate |
|---|---|---|---|---|---|---|---|---|---|
| | PLGA MW | | Solvent:polymer | Drug load | | release | at 4 weeks | at 8 weeks | at 12 weeks |
| Formulation | [kDa] | Solvent | ratio | [mg g⁻¹] | Injectable ^{a)} | burst [%]^{b)} | [μg day⁻¹] | [μg day⁻¹] | [μg day⁻¹] |
| RFB1 | 36.8 | DMSO | 4:1 | 128 | Y | 27.1 ± 6.3 | 2.4 ± 2.3 | 17.6 ± 4.4 | 16.1 ± 14.6 |
| RFB2 | 36.8 | DMSO | 2:1 | 109 | Y | 11.8 ± 2.0 | 5.0 ± 1.1 | 17.6 ± 16.2 | 10.9 ± 12.6 |
| RFB3 | 36.8 | NMP | 4:1 | 103 | Y | 32.2 ± 11.3 | 2.6 ± 3.0 | 23.7 ± 13.8 | 19.7 ± 13.3 |
| RFB4 | 36.8 | NMP | 2:1 | 87 | Y | 16.0 ± 5.4 | 6.0 ± 2.3 | 9.5 ± 5.7 | 17.4 ± 12.6 |
| RFB5 | 22.9 | DMSO | 4:1 | 128 | Y | 18.5 ± 2.5 | 16.3 ± 10.1 | 42.3 ± 25.9 | 3.9 ± 1.2 |
| RFB6 | 22.9 | DMSO | 2:1 | 109 | Y | 12.0 ± 2.4 | 9.5 ± 8.4 | 19.7 ± 2.6 | 3.5 ± 0.6 |
| RFB7 | 22.9 | NMP | 4:1 | 103 | Y | 24.2 ± 2.8 | 7.3 ± 5.0 | 42.9 ± 25.4 | 2.7 ± 4.7 |
| RFB8 | 22.9 | NMP | 2:1 | 87 | Y | 11.5 ± 3.7 | 2.5 ± 0.3 | 36.2 ± 5.0 | 13.8 ± 6.2 |
| RFB9 | 10.6 | DMSO | 4:1 | 127 | Y | 25.7 ± 5.5 | 69.2 ± 5.6 | 49.0 ± 25.9 | 5.9 ± 9.0 |
| RFB10 | 10.6 | DMSO | 2:1 | 109 | Y | 13.0 ± 1.6 | 59.9 ± 11.8 | 31.8 ± 5.4 | 33.0 ± 7.0 |
| RFB11^{c)} | 10.6 | NMP | 4:1 | 103 | Y | 46.6 ± 11.0 | 22.3 ± 5.3 | 18.5 ± 21.5 | N/A |
| RFB12 | 10.6 | NMP | 2:1 | 87 | Y | 19.5 ± 3.3 | 18.8 ± 4.5 | 14.2 ± 4.8 | 21.2 ± 7.7 |
| RFB13^{d)} | 10.6 | DMSO | 5:1 | 133 | Y | 22.7 ± 5.6 | 78.8 ± 18.9 | 18.2 ± 6.9 | N/A |

^{a)} Y = Injectable; ^{b)}Initial release burst is the cumulative percentage of RFB released into the release medium (PBS) 72 h after the injection; ^{c)}RFB11 implant was degraded after 9 weeks incubation; ^{d)}RFB13 implant was degraded at 8 weeks incubation; Data are expressed as mean ± SD.

All 13 formulations were injectable, solidified after injection into PBS, and released RFB for over eight weeks. Examples of LA-RFB formulation release profiles and corresponding daily release rates are shown in FIG. 2A,B. Release properties changed based on the material composition of the individual formulation. Specifically, the initial release burst was higher in formulations with a 4:1 solvent to polymer ratio than in formulations with a 2:1 solvent to polymer ratio (Table 3). RFB daily release rates at four weeks of incubation were significantly higher in formulations with low MW PLGA compared to formulations with higher MW PLGA (FIG. 2C). LA-RFB formulations with DMSO and a low MW PLGA (10.6 kDa) showed higher release rates at four weeks compared to formulations with NMP (Table 3, FIG. 2D). These results demonstrate that by manipulating the material composition of the ISFI formulation, release of drug from solidified implants can be modulated.^{[17]}

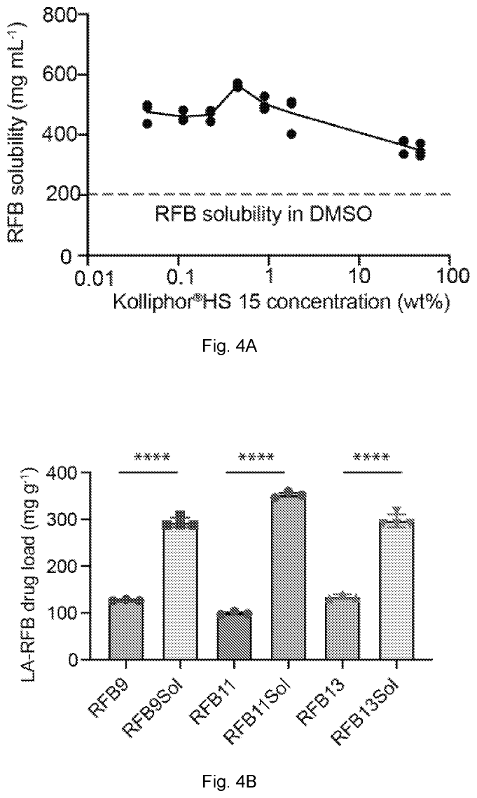

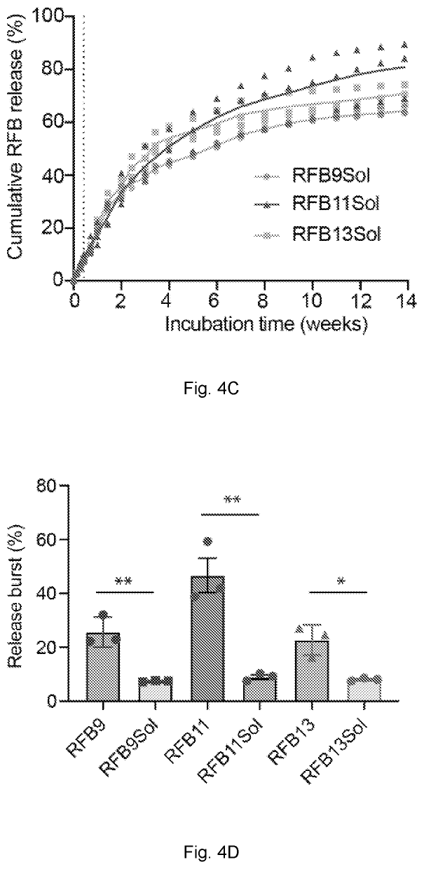

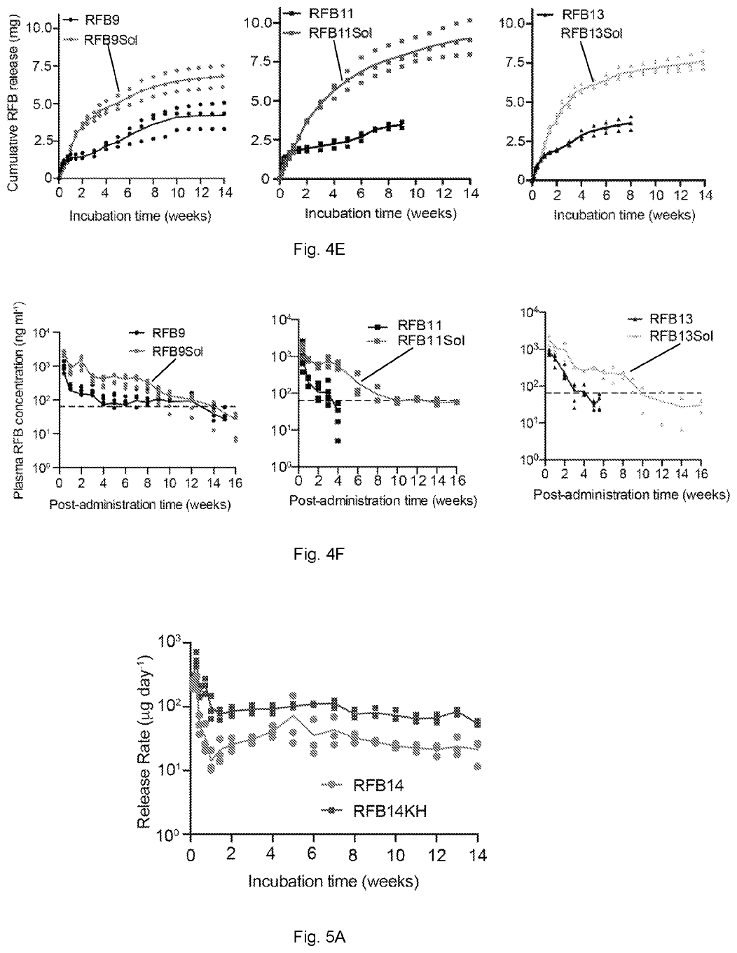

To further increase drug load in the LA formulations, drug solubility in a given solvent has to increase. Amphiphilic additives have previously been shown to improve drug solubility in water by trapping molecules and forming nanoscale aggregates; micelles.^{[37-40]} Even though DMSO and NMP are organic compounds, like water they are polar solvents. The addition of Kolliphor®HS 15, an amphiphilic nonionic surfactant formerly known as Solutol HS 15, to DMSO dramatically increased RFB solubility (FIG. 4A). The highest RFB solubility, 564±7 mg RFB per mL of DMSO (2.8 times higher than the RFB solubility in DMSO alone (202±22 mg mL⁻¹)) was achieved at 0.45% Kolliphor®HS 15. Similarly, the presence of Kolliphor®HS 15 in NMP also increased RFB solubility. The highest RFB solubility (702±10 mg mL⁻¹) was observed in NMP with 8.8% Kolliphor®HS 15, which is 4.7 times higher than the RFB solubility in NMP without Kolliphor®HS 15 (148±34 mg mL⁻¹) (FIG. 3A). RFB solubility in DMSO was also substantially improved in the presence of low concentrations of other amphiphilic compounds, including D-α-Tocopherol polyethylene glycol 1000 succinate (TPGS, FIG. 3B), Tween 20, Tween 80 (FIG. 3C), and Pluronic F127 and F68 (FIG. 3D).

To determine if the improved RFB solubility would result in increased RFB load in LA-RFB formulations, the material composition of the formulations with the highest release rates at four weeks of incubation (RFB9, RFB11, and RFB13, Table 3) were modified to include 0.45% Kolliphor®HS 15 in the DMSO-based formulations (RFB9 and RFB13) and 8.8% Kolliphor®HS 15 in the NMP-based formulation (RFB11) (Table 4). To indicate the presence of Kolliphor®HS 15 (formerly Solutol), new formulations were denoted RFB9Sol, RFB11Sol, and RFB13Sol. The RFB load in formulation RFB9Sol increased from 126.9±1.8 mg g⁻¹ (RFB9) to 293.4±10.2 mg g⁻¹, the RFB load in formulation RFB11Sol increased from 99.1±3.6 mg g⁻¹ (RFB11) to 352.4±6.8 mg g⁻¹, and the RFB load in formulation RFB13Sol increased from 134.3±5.3 mg g⁻¹ (RFB13) to 297.1±13.5 mg g⁻¹ (mean±SD, FIG. 4B). Each of the new formulations (RFB9Sol, RFB11Sol, and RFB13Sol) released RFB for at least 14 weeks in vitro (FIG. 4C). Notably, these formulations had a reduced initial release burst at 72 h (FIG. 4D). Specifically, RFB9Sol had an initial burst 3.5 times lower than RFB9 (P=0.0046), RFB11Sol had an initial release burst 5.2 times lower than RFB11 (P=0.0042), and RFB13Sol had a release burst 2.8 times lower than RFB13 (P=0.0111, FIG. 2d). Consistent with their increased drug load, formulations containing Kolliphor®HS 15 also resulted in an increased amount of RFB released (FIG. 4E). Cumulative release of RFB in vitro after eight weeks was 6104±652 μg RFB from RFB9Sol compared to 3630±848 μg from RFB9 (P=0.0160), 7639±835 μg from RFB11Sol compared to 3352±247 μg from RFB11 (P=0.0014), and 6930±356 μg RFB from RFB13Sol compared to 3667±430 μg RFB from RFB13 (P=0.0005).

TABLE 4
In vitro properties of LA-RFB formulations with Kolliphor ®HS 15 and acid-ending PLGA

| | | | | | | Initial | Release rate | Release rate | Release rate |
|---|---|---|---|---|---|---|---|---|---|
| | PLGA MW | | Solvent:polymer | Drug load | | release | at 4 weeks | at 8 weeks | at 12 weeks |
| Formulation | [kDa] | Solvent | ratio | [mg g⁻¹] | Injectable ^{a)} | burst [%]^{b)} | [μg day⁻¹] | [μg day⁻¹] | [μg day⁻¹] |
| RFB9Sol | 10.6 | DMSO with | 4:1 | 293 | Y | 7.4 ± 0.2 | 48.3 ± 16.2 | 34.5 ± 4.5 | 10.4 ± 2.6 |
| | | 0.45 wt % | | | | | | | |
| | | Solutol | | | | | | | |
| RFB11Sol | 10.6 | NMP with | 4:1 | 352 | Y | 9.0 ± 1.3 | 121.4 ± 35.7 | 44.5 ± 3.4 | 31.8 ± 15.2 |
| | | 8.8% Solutol | | | | | | | |
| RFB13Sol | 10.6 | DMSO with | 5:1 | 297 | Y | 8.1 ± 0.5 | 53.3 ± 2.4 | 23.4 ± 9.3 | 15.4 ± 8.4 |
| | | 0.45 wt % | | | | | | | |
| | | Solutol | | | | | | | |
| RFB9Sol = | 10.6 | DMSO with | 4:1 | 128 | Y | 34.4 ± 4.5 | 20.7 ± 4.6 | 30.3 ± 15.5 | N/A |
| 9^{c)} | | 0.45 wt % | | | | | | | |
| | | Soluto1 | | | | | | | |
| RFB14 | 13.5 (Acid- | DMSO | 4:1 | 130 | Y | 12.6 ± 0.9 | 47.5 ± 9.0 | 37.0 ± 4.8 | 24.5 ± 5.2 |
| | ending) | | | | | | | | |
| RFB14Sol | 13.5 (Acid- | DMSO with | 4:1 | 294 | Y | 9.6 ± 1.6 | 92.6 ± 8.7 | 77.7 ± 13.2 | 67.2 ± 5.7 |
| | ending) | 0.45 wt % | | | | | | | |
| | | Soluto1 | | | | | | | |
| RFB14Sol = | 13.5 (Acid- | DMSO with | 4:1 | 132 | Y | 18.8 ± 5.7 | 27.0 ± 3.6 | 59.0 ± 10.2 | 9.7 ± 16.8 |
| 14 | ending) | 0.45 wt % | | | | | | | |
| | | Solutol | | | | | | | |

^{a)} Y = Injectable;
^{b)}Initial release burst is the cumulative percentage of RFB released into the incubation medium (PBS) 72 h after the injection;
^{c)}RFB9Sol = 9 implant was degraded after 9 weeks incubation in the release medium; Data are expressed by mean ± SD

In order to determine if the in vitro observations described above regarding initial release burst, duration of drug release and total amount of drug released overtime translated into efficient in vivo drug delivery we used a well characterized in vivo model based on BALB/c mice. Specifically, mice were administered a single subcutaneous injection (50 L) of the indicated LA-RFB formulations and drug levels were monitored in plasma over time. Notably, plasma RFB concentrations were above the RFB MIC (64 ng mL⁻¹)^{[41, 42]} for longer periods of times in all the mice treated with the formulations containing Kolliphor®HS 15. The most remarkable differences were noted with formulations RFB11Sol and RFB13Sol that released drug for 6 and 9 weeks longer than the corresponding formulations without Kolliphor®HS 15 (FIG. 4F). However, even though the duration of drug delivery from formulation RFB9Sol was only extended by an additional two weeks, this formulation maintained plasma RFB concentrations above the MIC for the longest time and was therefore used for further optimization.

### Example 2

### Uncapped Acid-Ending PLGA Increases RFB Release Rates at Later Stages of RFB Delivery

The data presented above indicates an initial sustained release of RFB from RFB9Sol. However, release decreased over time resulting in a decrease in plasma RFB concentrations (FIG. 2f). Terminal stages of drug release from PLGA implants are primarily controlled by polymer biodegradation.^{[43, 44]} A delay in degradation time has been found for an ester end-capped PLGA in comparison with a more hydrophilic PLGA without ester capping (acid-ending) of a similar molecular weight and co-polymer composition.^{[30, 45, 46]} We therefore hypothesized that the use of uncapped acid-ending PLGA instead of ester end-capped PLGA would lead to increased release rates in vitro and higher drug concentrations at later time points. A new formulation, RFB14, was prepared with the same composition as RFB9, but with acid-ending PLGA (Supplemental Table 1). The RFB14 formulation demonstrated increased in vitro release rates at later time points compared to RFB9. For example, at twelve weeks RFB14 release rates were 24.5±5.2 μg per day, compared to RFB9 release rates of 5.9±9.0 μg per day (mean±SD, Supplemental Table 1). Similarly, RFB14Sol (a formulation with the same composition as RFB9Sol but with an acid-ending polymer) also had a higher in vitro release rate at 12 weeks compared to RFB9Sol (RFB14Sol: 67.2±5.7 μg per day, RFB9Sol: 10±2.6 μg per day, mean±SD). RFB14Sol had an increased release rate at all time points measured compared to RFB14 (FIG. 5A). Based on these encouraging results, we performed a pharmacokinetic analysis of these formulations in vivo using BALB/c mice. A single subcutaneous injection of 50 μL of RFB14 or RFB14Sol showed that the formulations based on an acid-ending PLGA delivered higher RFB plasma concentrations compared to RFB9 and RFB9Sol (FIG. 5B, FIG. 2f). At 16 weeks post administration, plasma RFB concentrations were three times higher in mice that received RFB14Sol (78.1±13.6 ng mL⁻¹, mean S.E.M.) compared to mice administered RFB9Sol (22.7±6.8 ng mL⁻¹, mean S.E.M.).

The efficiency of RFB penetration into tissues was assessed two and six weeks after a single 50 μL subcutaneous injection of RFB14Sol. RFB concentrations in organs susceptible to Mtb were analyzed by liquid chromatography followed by mass spectrometry (LC/MS/MS) (FIG. 5C). Consistent with sustained release of drug from the formulation, no differences were observed in RFB tissue concentrations analyzed at two weeks and six weeks after RFB14Sol administration (two weeks: lung 8.0±0.8 μg g⁻¹, liver 11.0±1.4 μg g⁻¹, spleen 9.3±1.2 μg g⁻¹, kidney 13.3±4.0 μg g⁻¹, lymph nodes 17.6±3.8 μg g⁻¹; six weeks: lung 6.7±1.6 μg g⁻¹, liver 7.8±2.1 μg g⁻¹, spleen 9.0±2.1 μg g⁻¹, kidney 8.3±1.7 μg g⁻¹, lymph nodes 14.0±3.2 μg g⁻¹; data are expressed as mean S.E.M., lung P=0.4951, liver P=0.2000, spleen P=0.8857, kidney P=0.2000, lymph nodes P=0.8857, Mann-Whitney U test). The mean tissue to plasma ratio at two weeks post administration was 16.4 in the lung, 20.5 in the liver, 20.1 in the spleen, 23.8 in the kidney, and 34.0 in the lymph nodes and was similar to the tissue to plasma ratio at six weeks post administration in tested organs (lung P=0.9999, liver P=0.8857, spleen P=0.4857, kidney P=0.9999, lymph nodes P=0.8857, Mann-Whitney U test), suggesting high RFB penetration to tissues most affected by Mtb infection (FIG. 5D). In conclusion, acid-ending PLGA formulations had superior in vitro and in vivo properties compared to formulations with ester-capped PLGA, resulting in long-term drug release with substantial tissue penetration.

### Example 3

### a Second Injection of RFB14Sol Administered 8 or 12 Weeks Later Provides Drug Delivery for Up to 36 Weeks

To extend RFB delivery beyond four months, in a separate experiment, a second dose of the same volume and composition of RFB14Sol was administered 8 or 12 weeks after the first injection, and plasma levels of RFB were monitored longitudinally (FIG. 5E). All mice showed similar plasma RFB concentrations after the first dose of RFB14Sol (compare panels b and e in FIG. 3). The booster injection rapidly increased plasma RFB concentrations back to the initial levels observed with the first dose, regardless of the timing of the booster. While mice that received one dose of RFB14Sol showed plasma RFB concentrations that gradually decreased over time to the RFB MIC by 18 weeks post administration (FIG. 3b), mice that received a booster maintained RFB plasma concentrations above the MIC for 36 weeks (FIG. 3e).

### Example 4

### Long-Term RFB Stability in LA-RFB

Having demonstrated the efficient long-term delivery of RFB we evaluated the long-term stability of the formulation. The stability of the RFB14Sol formulation was evaluated during storage at room temperature (25° C.) in the dark for changes in physical appearance, residual RFB concentration, and chemical integrity at 4, 6, 9, 12, 15, and 18 months. The residual RFB compared to the initial RFB concentration in RFB14Sol was 100.7±7.4% at 4 months, 99.0±0.4% at 6 months, 100.4±0.3% at 9 months, 96.5±1.0% at 12 months, 96±0.5% at 15 months, and 98.2±4.5% at 18 months (mean±SD) (FIG. 6A). HPLC histograms of the residual amount of RFB in RFB14Sol showed a single peak with similar retention times (5.4-5.7 min) for 18 months of storage (FIG. 6B). No change in physical appearance or consistency was observed. Interestingly, a solution of RFB in DMSO without polymer stored under the same conditions as RFB14Sol had 67% residual RFB after 3 months, 53% after 4 months, and 25% after 6 months of storage, suggesting faster degradation of RFB in DMSO compared to RFB14Sol (FIG. 6C). These results indicate that the RFB14Sol formulation is stable at room temperature for at least 18 months.

### Example 5

Increased Drug Load Improves RFB Release Kinetics from LA-RFB Formulations

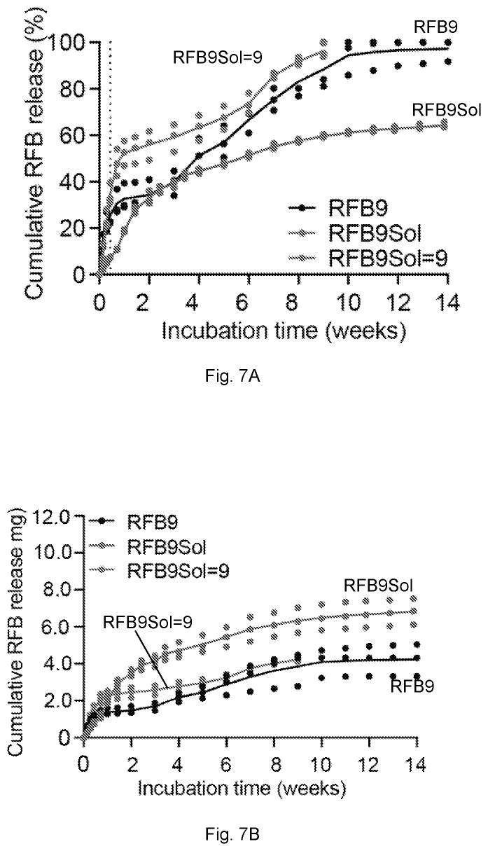

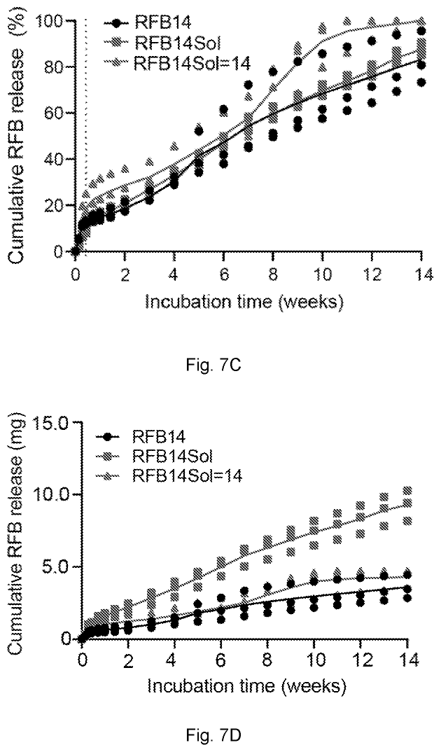

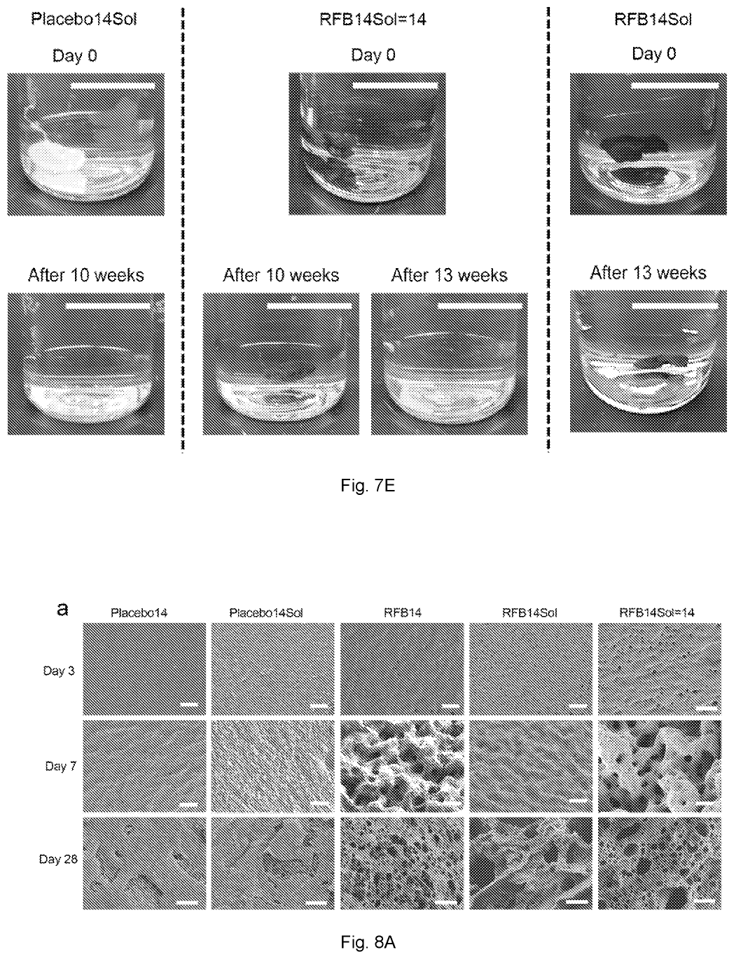

We showed that the addition of amphiphilic additives to LA-RFB formulations allows higher drug load and dramatically improved RFB release kinetics (FIG. 2, Supplemental Table 1). To distinguish whether Kolliphor®HS 15 or the increased drug load contributed to the improved RFB release kinetics, new LA-RFB formulations were prepared. Formulations RFB9Sol=9 and RFB14Sol=14 had the same compositions and drug loads as formulations RFB9 and RFB14, respectively, but contain Kolliphor®HS 15. “=9” or “=14” at the end of the name of the formulations containing Kolliphor®HS 15 is used to indicate that they contain the same amount of RFB as in the original RFB9 and RFB14 formulations. Therefore, any differences observed between formulations RFB9Sol=9 and RFB9 (128 mg g⁻¹ and 127 mg g⁻¹ RFB, respectively) or RFB14Sol=14 and RFB14 (132 mg g⁻¹ and 130 mg g⁻¹, respectively) that have the same drug load can be attributed to the presence of Kolliphor®HS 15. Additionally, any differences observed between formulations RFB9Sol=9 and RFB9Sol (128 mg g⁻¹ and 293 mg g⁻¹, respectively) or RFB14Sol=14 and RFB14Sol (132 mg g⁻¹ and 294 mg g⁻¹, respectively) can be attributed to differences in RFB load. RFB9Sol=9 demonstrated a high initial release burst (34.4±4.5%, mean±SD) compared to RFB9 (25.7±5.5%, P=0.0043) and RFB9Sol (7.4±0.2%, P=0.0002, one-way ANOVA) (FIG. 7A). RFB9Sol=9 released a similar amount of RFB to RFB9 over time but less than RFB9Sol (FIG. 7B). Specifically, at 8 weeks of in vitro incubation, cumulative release of RFB from RFB9Sol=9 was 4218±135 μg, RFB9 3860±933 μg (P=0.7242), and RFB9Sol 6328±681 μg of RFB (mean+SD, P=0.0148, RFB9Sol compared to RFB9Sol=9; P=0.0065, RFB9Sol compared to RFB9, one-way ANOVA). RFB14Sol=14 showed a similar initial release burst of RFB compared to RFB14 that was higher when compared to the release rate of RFB14Sol (18.8±5.7%, 9.6±1.6%, and 12.6±0.9%, respectively; FIG. 7C and Supplemental Table 1, P=0.0406, RFB14Sol=14 compared to RFB14Sol, one-way ANOVA). After 12 weeks of incubation in PBS, cumulative release of RFB from RFB14Sol was higher compared to RFB14Sol=14 (P=0.002) and RFB14 (P=0.003) (RFB14: 3753±924 μg, RFB14Sol=14: 3274±50 μg, RFB14Sol: 8371±616 μg, one-way ANOVA) (FIG. 7D). These results indicate that the addition of Kolliphor®HS 15 to the formulation increased the initial release burst, while the high drug load in the formulation resulted in sustained and increased RFB release over a long period of time.

### Example 6

### Increased Drug Load Decreases Implant Erosion

To further investigate the basis for the extended release from formulation RFB14Sol, we evaluated implant erosion in vitro. Interestingly, we noted that implant erosion in vitro decreased inversely to the drug load present in the implant. Placebo14Sol with acid-ending PLGA and Kolliphor®HS 15 had the same composition as RFB14Sol but did not contain RFB (placebo14Sol, 13.5 kDa acid-ending PLGA, 0.45 wt % Kolliphor®HS 15 in DMSO, 4:1 DMSO:PLGA ratio). Placebo14Sol formed implants that took 10 weeks to fully dissipate in vitro (FIG. 7E). Implants formed from RFB14Sol=14 that had the same composition as placebo14Sol and contained 132±6 mg g⁻¹ RFB completely dissipated by 13 weeks of incubation in PBS (FIG. 4e). Notably, implants formed from RFB14Sol with 294 mg g⁻¹ RFB were not eroded by 13 weeks (last time evaluated). Dissipation results suggest that higher drug load in the implant result in slower polymer erosion allowing for prolonged release of RFB from the implant.

### Example 7

### Effect of Kolliphor®HS 15 and Drug Load on LA-RFB Implant Structure

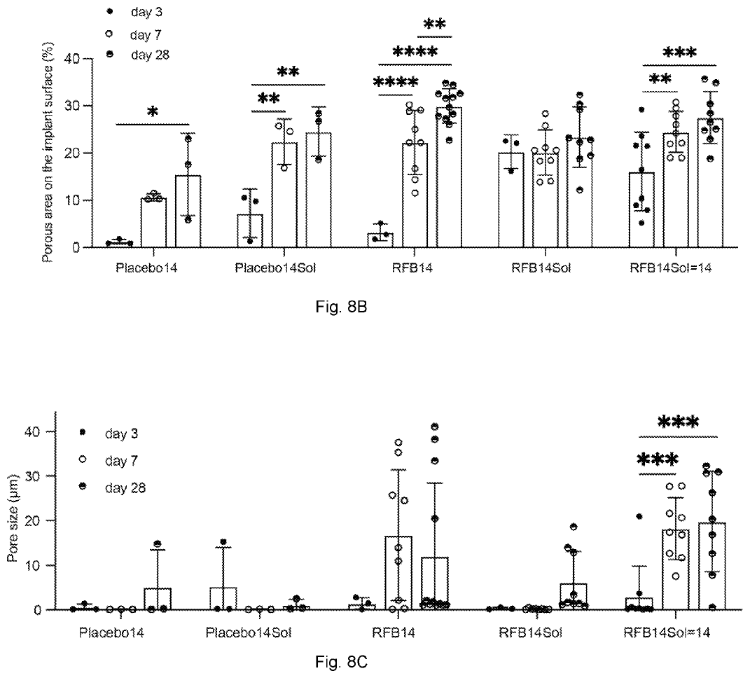

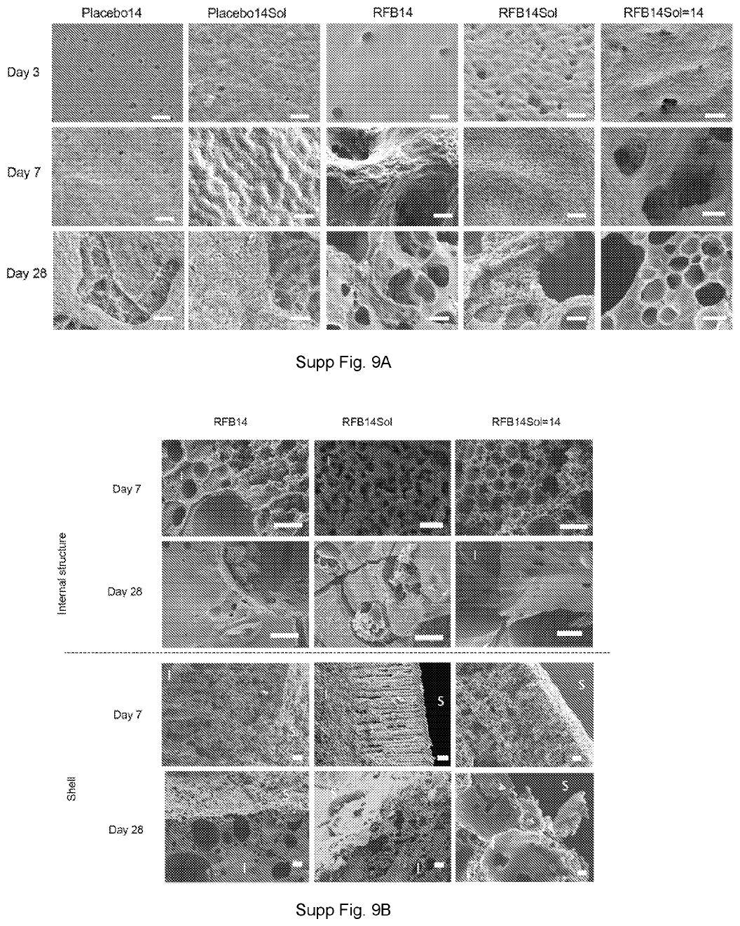

The kinetics of drug release from ISFI formulations are influenced by implant structure^{[20, 47]} which is determined by multiple factors, including the material composition of the formulation (polymer type, solvent, additives, and drug properties), the rate of phase inversion, the injection site, and polymer degradation.^{[18, 48-54]} Biodegradable ISFI polymer implants have a porous microstructure with an interconnected network of pores that allows diffusion of water into the implant and its bulk erosion.^{[52]} Pore size and the porous area on the surface of the implant determine water access and its uptake facilitating polymer degradation and implant erosion.^{[55-57]} To further understand the basis for the improved release kinetics of RFB14Sol, both surface and freeze fractured cross-sections of implants with different RFB load and composition and their corresponding drug-free placebo formulations were analyzed using scanning electron microscopy (SEM).¹⁶ Solidified implants of RFB14 (130 mg g⁻¹ RFB), RFB14Sol=14 (132 mg g⁻¹ RFB), RFB14Sol (294 mg g⁻¹ RFB), placebo14 with the same composition as RFB14 but without drug (13.5 kDa acid-ending PLGA, 4:1 DMSO:PLGA ratio), and placebo14Sol with the same composition as RFB14Sol and RFB14Sol=14 but without RFB (see paragraph 2.6 and Supplemental Table1) were collected after three days of incubation in PBS (i.e. the end of the solidification process^{[17]}) and after 7 and 28 days of incubation in PBS. SEM images of the implant surface (FIG. 8A, FIG. 9A for higher magnification) were evaluated for porous area (defined as % of surface area filled with pores) (FIG. 8B) and pore size (m) (FIG. 8C). Both placebo14 and placebo14Sol had low porous area (1.2±0.5 and 7.2±5.1%, respectively) that increased significantly over time (day 7: 10.6±0.7% and 22.4±4.8%, day 28: 15.5±8.8% and 24.5±5.2%, respectively) (FIG. 8B). Placebo implants had small pores (0.6±0.7 μm and 5.2±8.7 μm, respectively) which did not change during incubation (FIG. 5c). Pore sizes were similar between the placebo implants with and without Kolliphor®HS 15.

RFB14 implants with 130 mg g⁻¹ RFB load and no Kolliphor®HS 15 had a low porous area (3.2±1.8%) that significantly increased over time (22.2±6.8% at 7 days and 30.0±3.6% at 28 days, FIG. 5b). The presence of Kolliphor®HS 15 in LA-RFB formulations increased the initial porous area (RFB14Sol: 20.3±3.6% and RFB14Sol=14: 16.1±9.7% vs. RFB14: 3.2±1.8%). Implants with high RFB load (RFB14Sol, 294 mg g⁻¹ RFB) had no significant increase in porous area during the incubation period (RFB14Sol: 20.3±3.6% at 3 days, 20.1±4.8% at 7 days, and 23.4±6.4% at 28 days). These data indicate that the presence of Kolliphor®HS 15 in the formulation results in increased porous area on the implant surface after solidification (indicated as day 3 in panel b, FIG. 5). In addition, these data also indicate that increased drug load prevents further increases in porous area during incubation (indicated as days 7 and 28 in panel b, FIG. 5).

Pore size on the surface of RFB14 and RFB14Sol=14 implants increased during implant erosion (from 1.4±1.3 μm and 3.0±6.8 μm at 3 days to 16.7±14.7 μm and 18.2±7.0 μm at 7 days, and 29.6±4 μm and 27.5±5.5 μm at 28 days, mean+SD). These increases in pore size did not occur during the first 7 days for the implants with high drug load and were significantly less after 28 days of incubation (RFB14Sol: pore size 0.3±0.2 μm at 3 days, 0.2±0.1 μm at 7 days, and 6.1±7.0 μm at 28 days of incubation, FIG. 5c). These results suggests that a high drug load slows implant erosion and stabilizes the implant, contributing to extended drug release.

The internal structure of LA-RFB implants had a typical honeycomb-like structure with uniform macro-voids and interconnected pores after seven days of incubation (FIG. 9B).^{[53, 58]} Addition of Kolliphor®HS 15 resulted in increased asymmetry and disorganization of the internal implant structure (RFB14 vs. RFB14Sol=14). High drug load in the implant resulted in a more organized internal structure, with fewer macro-voids and interconnected pores (RFB14Sol=14 vs. RFB14Sol at seven days of incubation). The internal structure of the implants for all LA-RFB formulations (RFB14, RFB14Sol, and RFB14Sol=14) became less organized over time (Supplemental FIG. 4b). Cross-sections of the LA-RFB implants showed a defined shell at the implant surface with a more dense and organized structure than the internal structures of the implant. The shell was most distinct in implants with high drug load (RFB14Sol), where it had highly organized fingerlike pores directly connecting the surface and internal parts of the implant (Supplemental FIG. 4b).^{[59]} Shells of implants with the same composition but with lower drug load were less organized than in implants with higher drug load, regardless of the presence of Kolliphor®HS 15 (RFB14 and RFB14Sol=14). By 28 days of incubation, the shells eroded and were no longer visible for all LA-RFB implants. Together, these results show that the presence of Kolliphor®HS 15 in LA-RFB formulations resulted in a large porous surface area after implant formation that facilitates water diffusion into the implant and faster polymer degradation and erosion of the implant.^{[55]} High drug load slowed implant erosion despite the presence of Kolliphor®HS 15. Therefore, drug load in formulations containing Kolliphor®HS 15 has a large impact on modulating RFB release kinetics.

### Example 8

In Vivo Efficacy of LA-RFB Against Mycobacterium tuberculosis

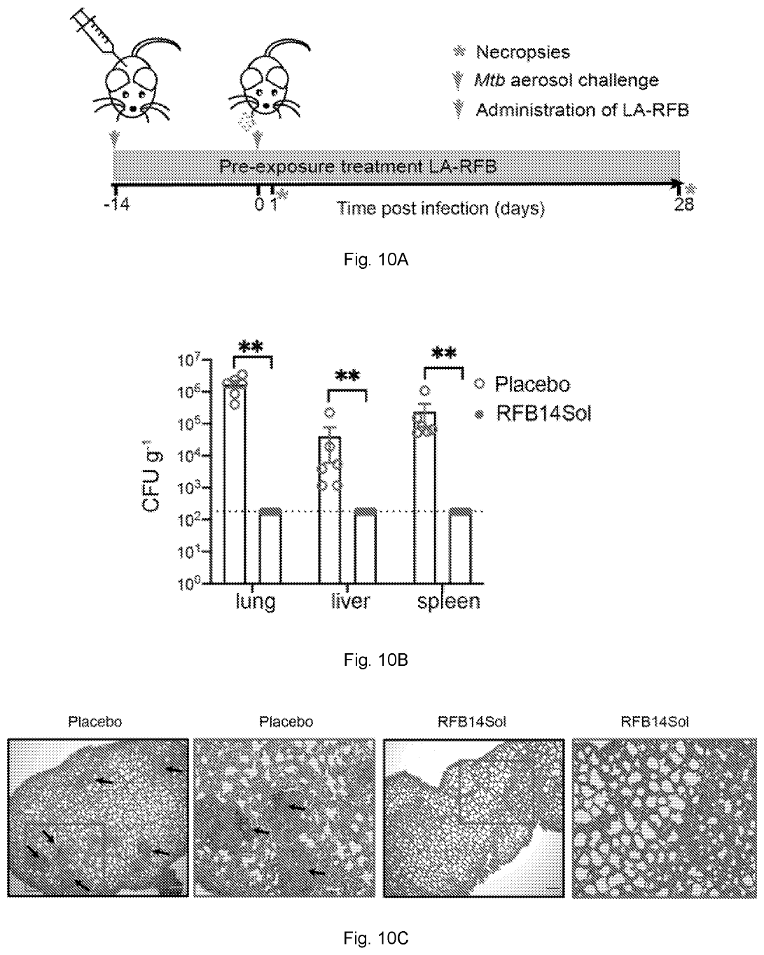

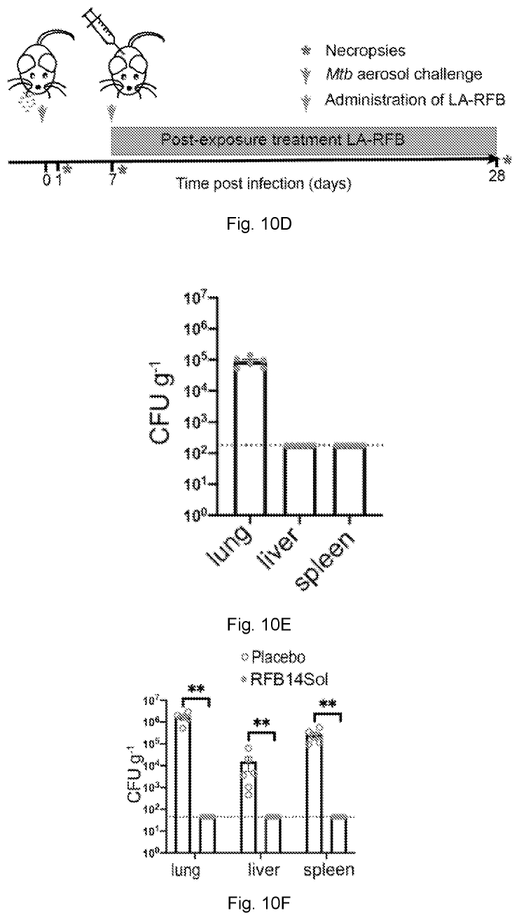

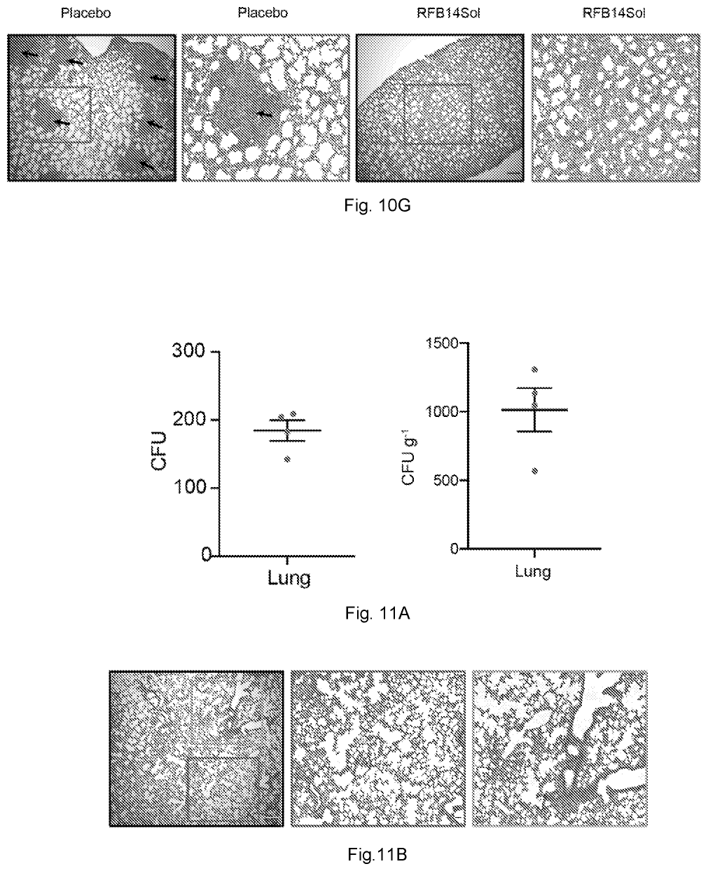

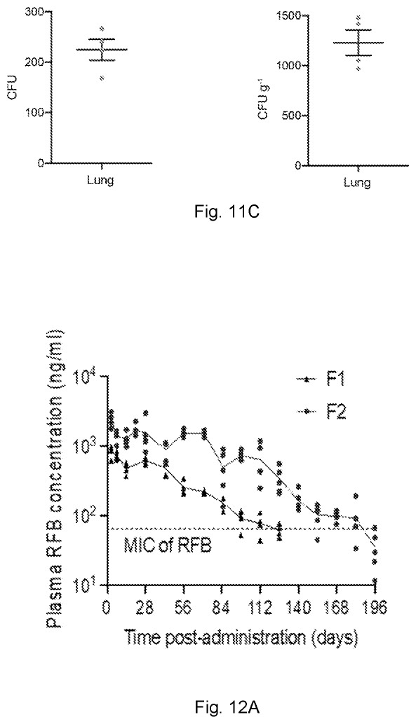

The optimized LA-RFB formulation (RFB14Sol) was tested in two in vivo experiments to determine its efficacy against Mtb infection. First, we tested the ability of RFB14Sol to prevent initial Mtb infection in BALB/c mice when administered as pre-exposure prophylaxis (FIG. 10A). For this, BALB/c mice were treated with a single subcutaneous injection (50 μL) of placebo (n=6) or RFB14Sol (n=6). Two weeks later, both groups of mice were exposed to the virulent Erdman strain of Mtb via aerosol delivery. At this time, four additional untreated mice were also exposed to aerosolized Mtb to determine the absolute dose of Mtb delivered to the lung. By homogenizing and plating out the entirety of the lung (approximately 0.2 grams of tissue) of these four animals one day post-aerosol exposure, the infectious dose delivered was determined to be 185±15 CFU Mtb (or 1013±158 CFU per gram of lung tissue) (FIG. 11A). Four weeks after Mtb exposure, placebo and RFB14Sol treated mice were necropsied, and the lung, liver, and spleen were analyzed for bacterial burden. Placebo treated mice exhibited a more than 3-log increase in bacterial burden in the lung over time (1.8×10⁶±4.4×10⁵ CFU g⁻¹, mean±S.E.M.) and substantial dissemination to distal organs including liver (4.2×10⁴±3.6×10⁴ CFU g⁻¹, mean±S.E.M.) and spleen (2.5×10⁵±1.7×10⁵ CFU g⁻¹ mean+S.E.M) (FIG. 10B). The lungs of placebo treated mice also exhibited gross pathological changes characterized by altered lung structures, immune infiltrates, thickened alveolar walls, and disorganized granulomatous lesions that are consistent with Mtb infection and typical of mice infected with Mtb (FIG. 10C).^{[60]} In contrast, RFB14Sol treated mice had no detectible bacterial burden or any pathology associated with Mtb infection in any organ analyzed (FIG. 6b-c). Further, lungs from mice that received RFB14Sol and were Mtb infected were not visibly different from uninfected mice that also received RFB14Sol (FIG. 11B). These results show that pre-exposure prophylaxis with a single subcutaneous injection of RFB14Sol efficiently prevents Mtb infection and the development of its associated pathology.

Having established the ability of RFB14Sol to prevent infection, we also used BALB/c mice to assess its ability to control acute Mtb infection (FIG. 10D). For this purpose, BALB/c mice (n=22) were exposed to Mtb via aerosol. The Mtb dose delivered in this experiment was determined 24 hours later in four mice (225±21 CFU per mouse, 1229±128 CFU per gram of lung tissue, FIG. 11C). One week after Mtb exposure, bacterial burden in the lungs, liver, and spleen in 6 of the 22 mice was assessed. At this time, all 6 mice analyzed had detectable bacterial burden in the lungs (8.8×10⁵±1.4×10⁴ CFU g⁻¹, mean±S.E.M). At this time point there was no detectible bacterial burden in the liver or spleen (FIG. 10E). The remaining mice were treated with a single 50 μL subcutaneous injection of either placebo (n=6) or RFB14Sol (n=6). Three weeks after treatment initiation (four weeks after Mtb exposure), placebo and RFB14Sol treated mice were necropsied, and the lungs, liver, and spleen were analyzed for bacterial burden and pathological manifestations of Mtb infection. Placebo treated mice had Mtb in all organs analyzed (1.7×10⁶±3.2×10⁵ CFU g⁻¹ lung; 1.5×10⁴±9.7×10³ CFU g⁻¹ liver; 2.6×10⁵±7.2×10⁴ CFU g⁻¹ spleen, mean±S.E.M., FIG. 10F). Pathological changes consistent with Mtb disease including granulomatous lesions were observed on stained lung sections from all placebo treated mice (FIG. 10G). In contrast, no Mtb was detected in mice treated with RFB14Sol and no pathological changes in tissues were noted (FIG. 6f,g). Therefore, a single injection of RFB14Sol administered one week after infection was able to efficiently reduce bacterial burden in the lung and to prevent Mtb dissemination to distal organs in mice exposed to Mtb.

### Example 9

Changes to the Preparation Process that Leads to Increased Drug Load.

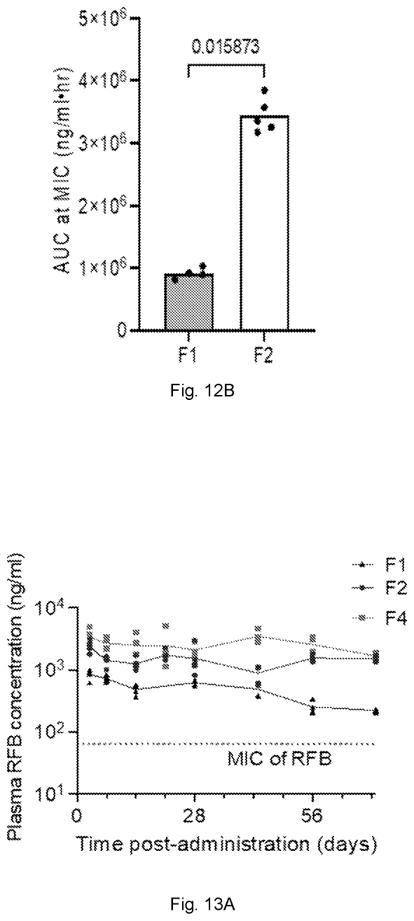

We were able to increase the drug load in formulations by a change in the preparation process. Specifically, we changed the order in which components of the ISFI are added. Instead of solubilizing the drug in solvent and then adding the polymer, we first, solubilize the polymer PLGA (13.5 kDa, LA:GA=50:50) in DMSO containing 0.45% Kolliphor®HS 15 at a 4:1 DMSO:polymer ratio (the solvent composition previously optimized for formulation F1). We then were able to add a greater amount of RFB resulting in a new formulation containing 400 mg/g RFB (designated formulation 2 or F2). FIG. 12 shows a pharmacokinetic profile of formulation F2 in BALB/c mice. RFB-ISFI formulation F2 delivered higher plasma concentration of RFB for an additional 2 months compared to formulation F1 (FIG. 12A). The amount of RFB from initial administration to time when plasma concentration reached MIC was 3 times higher in mice injected with formulation F2 compared to formulation F1. No overt inflammatory or adverse reactions were noted with higher drug load in new formulation.

### Example 10

Hydrophobic Additives Increase Drug Release from Implants.

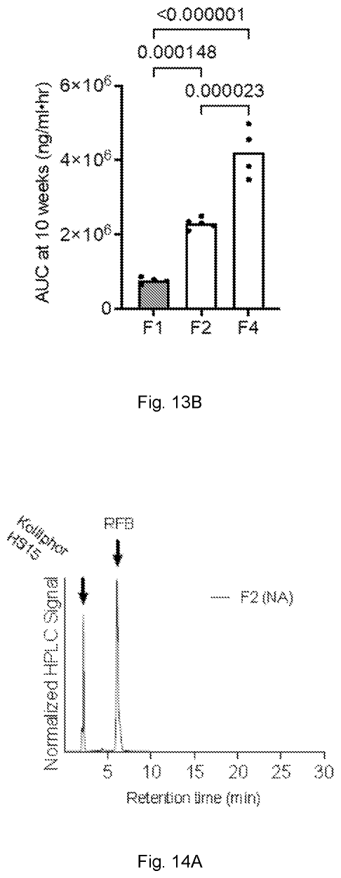

The effect of hydrophobic additives on drug release was evaluated. We discovered that compounds with higher hydrophobicity (log P) than the drug present in the formulation can increase release of the drug from the implant (log P of drug <log P of hydrophobic additive). Specifically, we evaluated oleic acid (OA), an FDA approved inactive hydrophobic (log P=6.5) additive.^{[61]} DMSO with three different concentrations of oleic acid (6.4 wt %, 12.0 wt %, and 21.4 wt %) and 0.45% Kolliphor®HS 15 were used as solvent, PLGA: solvent ratio was 1:4 and RFB load 400 mg/g. All three formulations were injectable with a 19 G needle and created implants after injection into aqueous buffer (PBS). Release properties were evaluated in vitro and in vivo. Analysis in BALB/c mice showed increased plasma concentration in mice treated with formulation containing OA compared to F1 and F2. (FIGS. 13A and B).

Similar effect on release of the drug from the implants can be obtained with other hydrophobic compounds with Log P higher than the drug in formulation in concentration up to 35% w.

Examples of such hydrophobic additives:
saturated, monounsaturated, and polyunsaturated fatty acids: oleic acid, stearic acid, arachidic acid, palmitic acid, linolic acid, myristic acid, α- or β-eleostearic acid, 9,11-octadecadienoic acid, eicosapentaenoic acid
fatty alcohols: hexacosanol, octacostanol, dotriacontanol
terpenes: nerolidol, farnesol
sterols: cholesterol, sitosterol, stigmasterol, stigmastanol,
tocopherols: vitamin E and its derivative

### Example 11

### Terminal Sterilization of ISFI Formulation by Autoclaving

Terminal sterilization is a preferred method to obtain a sterile drug product and an important step in formulation development. We evaluated formulations F2 and F4 for terminal sterilization by autoclaving. Maximum temperature tested was 121° C. for 20 min. Formulations were prepared in glass vials that were sealed before autoclaving.

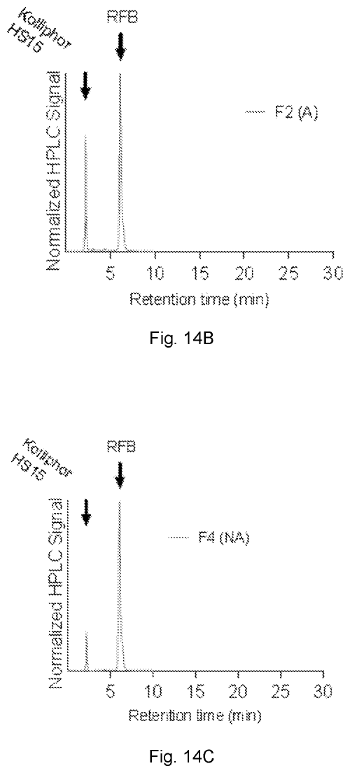

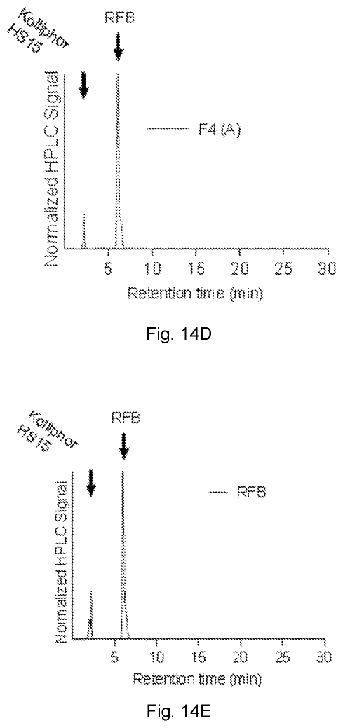

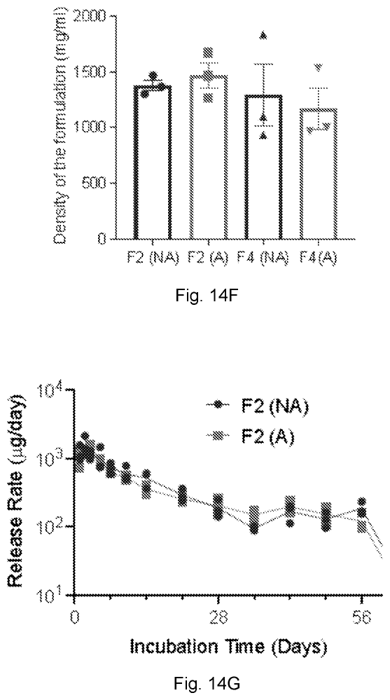

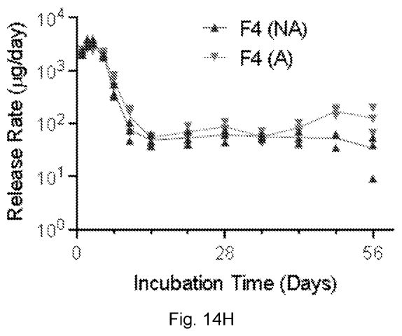

Properties of formulations including chemical identity of RFB analyzed by HPLC (FIG. 14A-E), density (FIG. 14F), and in vitro release properties (FIG. 14G-H) were assessed after autoclaving and compared to non-autoclaved formulations. Tested quality attributes were not affected by the terminal sterilization.

### Discussion of Examples 1-11

Drug-resistant TB can arise when Mtb is exposed to subtherapeutic anti-TB drug concentrations for extended periods of time.^{[62]} This is often associated with low adherence to treatment regimens. The use of LA drug formulations is a promising strategy that can improve treatment adherence.^{[63]} The most important factor in the development of effective LA formulations is the daily drug release (drug release rate) that leads to sufficient plasma concentration to inhibit Mtb in vivo.

The material composition of ISFI formulations allows for extensive optimization of release rates by changes in their material composition. Analysis of 13 different ISFI formulations of RFB (Table1) showed three formulations with high release rates in vitro after one month of incubation based on PLGA and DMSO as solvent (RFB 9, RFB10, and RFB13). Indeed, statistical analysis showed significantly higher release rates in formulations with low MW polymer (i.e. 10.6 kDa) compared to formulations with high MW polymer (i.e. 22.9 and 36.8 kDa) (FIG. 2C) and in formulations containing DMSO compared to those containing NMP as solvent (FIG. 2D). However, formulations with high daily release rates also require high drug load to maintain sustained drug release for long periods of time. Therefore, a high drug load in the formulation is an additional important factor during LA formulation development.

Amphiphilic additives Kolliphor®HS 15, TPGS, Tween 80, Tween 20, and Pluronic F68 and F127 were able to dramatically increase the solubility of RFB in DMSO and NMP (FIG. 4A and FIG. 3) and allowed for significantly increased drug loads in RFB formulations (FIG. 4B). Formulations with amphiphilic additives and high drug load had 1) decreased initial release bursts (FIG. 4D) and 2) extended drug release in vitro (FIG. 4C, E) resulting in 3) increased plasma concentrations for extended periods of time (FIG. 4F). These improvements could be explained either by increased drug load or by the presence of amphiphilic additives.^{[19, 64]} To distinguish between these two possibilities, formulations with Kolliphor®HS 15 and low drug loads RFB9Sol=9 and RFB14Sol=14 were prepared. These formulations contained amphiphilic additives but the same composition and RFB loads as in formulations RFB9 and RFB14. Therefore, differences in properties between RFB9Sol=9 versus RFB9 and RFB9Sol=14 versus RFB14 could be attributed solely to the presence of amphiphilic additives. Formulations with amphiphilic additives (RFB9Sol=9 and RFB14Sol=14) showed faster cumulative drug release compared to RFB9 and RFB14. The increase was achieved mainly by an increased release burst. This is consistent with a previous report showing that the amphiphilic additive Tween 80 increased release of buprenorphine hydrochloride by increasing the initial burst with a minimal effect on later phases of drug delivery.^{[64]} Thus, the lower release burst and extended RFB release observed in formulation RFB14Sol is caused by the increase in drug load rather than the presence of amphiphilic additives. Biodegradable implants degrade by bulk degradation that depends on diffusion of water into the implant.^{[65, 66]} Degradation can be slowed by suppressing water invasion into the implant by increasing implant hydrophobicity. Therefore, implants with higher load of a hydrophobic drug are expected to have higher hydrophobicity and slower implant erosion.^{[67, 68]} Indeed, we showed that RFB14Sol implants with a high drug load (294 mg g⁻¹ RFB) had slower erosion than similar placebo implants or implants with a lower drug load (130 mg g⁻¹ RFB) (FIG. 7E).

To further explore how the higher drug load decreases the initial release burst, we evaluated the surface of implants solidified from RFB14, RFB14Sol=14, and RFB14Sol formulations for porous area and pore size. Interestingly, solidified implants from RFB14 and RFB14Sol=14 that differ only by the presence of Kolliphor®HS 15 in RFB14Sol=14 showed very similar changes in porous area and pore size during implant erosion (FIG. 5). However, at 3 days of incubation (end of implant solidification^{[17]}), implants from the formulation with low drug load and Kolliphor®HS 15 (RFB14Sol=14) have a larger porous area compared to implants with the same drug load without Kolliphor®HS 15 (RFB14). Because the pore size at 3 days of incubation is similar between formulations, the higher porous area of RFB14Sol=14 could be attributed to an increase in the number of pores present in the implant (FIG. 8A). This is consistent with the higher release burst in RFB14Sol=14, as more pores leads to increased water invasion into the implant and faster release of the drug. Interestingly, implants from RFB14Sol, which had both Kolliphor®HS 15 and a high drug load, also has higher porous area and more pores than RFB14 at 3 days of incubation, but contrary to RFB14Sol=14 had a lower release burst. While porous area and pore size increased significantly in RFB14Sol=14 throughout the implant erosion process, RFB14Sol had minimal changes in porous area and pore size for the first 28 days of incubation. It is thus possible that a higher load of hydrophobic drug in the formulation resulted in protection of the implant from erosion as well as a reduced initial release burst.

Subcutaneous administration of RFB14Sol (50 μL, drug load 294 mg g⁻¹ RFB) to BALB/c mice resulted in plasma concentrations of RFB that were 10 times higher than the MIC for at least four weeks post administration and that were above the MIC for 16 weeks post administration (FIG. 8B). Administration of the LA-RFB formulation resulted in high penetration of drug into tissues. The mean tissue to plasma ratio for the lung was 16.4 and for the spleen 20.1 (FIG. 5C). These high plasma concentrations and especially high tissue penetration are promising properties of the LA-RFB formulation that would allow translation to larger animals and humans. In comparison, daily oral administration of RFB (10 mg kg⁻¹ day⁻¹) resulted in a mean tissue to plasma ratio of 3.3 for the lung and 1.74 for the spleen.^{[69-71]} Preliminary estimates using empiric conversion based on body surface area between mice and non-human primates or humans (3.1 and 12.3 respectively^{[72, 73]}) confirm the feasibility of the use of <500 μL and <2 mL injections, respectively that could provide adequate drug level for at least one month.^{[72, 74-76]}

It has been shown that repeated dosing or prolonged exposure to rifampin, a member of the rifamycin family of drugs, can cause a decrease in plasma drug concentration by accelerating drug clearance via induction of cytochrome P450 and P-glycoprotein.^{[77]} RFB is a significantly weaker inducer of these metabolizing enzymes than rifampin and rifapentine.^{[28]} Indeed, repeated doses of LA-RFB at two or three months after administration of the first dose showed a similar pharmacokinetic profile (FIG. 3e), suggesting that prolonged exposure to RFB does not result in reduced plasma concentrations. Therefore, repeated subcutaneous dosing of LA-RFB can be used to substantially extend the duration of sufficient plasma RFB levels to inhibit Mtb. Importantly, due to the lower induction of cytochrome P450 and P-glycoprotein, RFB can be used in combination with many antiretroviral drugs commonly used for HIV treatment, making LA-RFB formulations attractive for LTBI treatment in people living with HIV (PLWH).

The standard of care for active TB infection requires multidrug therapy.^{[4, 9]} However, the WHO strategy for ending TB includes prevention, wherein a single anti tuberculosis drug is sufficient. In a model for pre-exposure prophylaxis, RFB14Sol prevented initial Mtb infection from occurring. Furthermore, in a model of acute Mtb infection, RFB14Sol successfully cleared Mtb infection from the lung and prevented its dissemination to distal organs. Importantly, granulomatous lesions which are normally associated with Mtb infection in mice were not observed in any animal that received RFB14Sol.

Taken together, we successfully developed a long-acting RFB formulation that solidifies into an implant upon subcutaneous injection. Manipulation of its material composition (drug load, additives, and type of polymer) provides control over the implant's microstructure, erosion, and the steady release of drug. LA-RFB showed high efficacy in pre-exposure and post-exposure prophylaxis mouse models, making this approach a promising strategy for further pre-clinical development. These results represent a significant advance in material composition of ISFI formulations with high clinical relevance.

### REFERENCES

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
[1]M. Pai, M. A. Behr, D. Dowdy, K. Dheda, M. Divangahi, C. C. Boehme, A. Ginsberg, S. Swaminathan, M. Spigelman, H. Getahun, D. Menzies, M. Raviglione, Nat Rev Dis Primers 2016, 2, 16076.
[2]R. M. G. J. Houben, P. J. Dodd, PLoS Med. 2016, 13.
[3]L. C. du Toit, V. Pillay, M. P. Danckwerts, Respir. Res. 2006, 7, 118.
[4] WHO, Global tuberculosis report 2021, 2021, 2021
[5]A. Verma, M. Kaur, L. V. Singh, D. Aggarwal, I. Verma, B. D. Radotra, S. Sharma, Sci. Rep. 2021, 11.
[6]S. Swindells, M. Siccardi, S. E. Barrett, D. B. Olsen, J. A. Grobler, A. T. Podany, E. Nuermberger, P. Kim, C. E. Barry, A. Owen, D. Hazuda, C. Flexner, Int J Tuberc Lung Dis 2018, 22, 125.
[7]S. T. Partnership, The potential impact of the COVID-19 response on tuberculosis in high-burden countries: modeling analysis, 2020, 2021
[8] WHO, Implementing the WHO Stop TB Strategy: A Handbook for National Tuberculosis Control Programmes., 2008.
[9] WHO, Guidelines for treatment of drug-susceptible tuberculosis and patient care, 2017 update, WHO, 2017.
[10]P. Kulkami, S. Akarte, R. Mankeshwar, J. Bhawalkar, A. Banerjee, A. Kulkarni, Ann Med Health Sci Res 2013, 3, 67.
[11]A. Zegeye, G. Dessie, F. Wagnew, A. Gebrie, S. M. S. Islam, B. Tesfaye, D. Kiross, PLoS One 2019, 14, e0210422.
[12]A. Kaushik, N. C. Ammerman, S. Tyagi, V. Saini, I. Vervoort, S. Lachau-Durand, E. Nuermberger, K. Andries, Antimicrob Agents Chemother 2019, 63.
[13]D. Havlir, M. Gandhi, Current opinion in HIV and AIDS 2015, 10, 282.
[14]E. N. Titus-Lay, E. D. Ansara, A. N. Isaacs, C. A. Ott, The mental health clinician 2018, 8, 56.
[15]S. Kempe, K. Mader, J. Control. Release 2012, 161, 668.
[16]M. Kovarova, S. R. Benhabbour, I. Massud, R. A. Spagnuolo, B. Skinner, C. E. Baker, C. Sykes, K. R. Mollan, A. D. M. Kashuba, J. G. Garcia-Lerma, R. J. Mumper, J. V. Garcia, Nature communications 2018, 9, 4156.
[17]S. R. Benhabbour, M. Kovarova, C. Jones, D. J. Copeland, R. Shrivastava, M. D. Swanson, C. Sykes, P. T. Ho, M. L. Cottrell, A. Sridharan, S. M. Fix, O. Thayer, J. M. Long, D. J. Hazuda, P. A. Dayton, R. J. Mumper, A. D. M. Kashuba, J. Victor Garcia, Nature communications 2019, 10, 4324.
[18]M. Parent, C. Nouvel, M. Koerber, A. Sapin, P. Maincent, A. Boudier, J. Control. Release 2013, 172, 292.
[19]P. D. Graham, K. J. Brodbeck, A. J. McHugh, J Control. Release 1999, 58, 233.
[20]K. J. Brodbeck, J. R. DesNoyer, A. J. McHugh, J. Control. Release 1999, 62, 333.
[21]C. Sekaggya-Wiltshire, K. E. Dooley, Expert Opin. DrugMetab. Toxicol. 2019, 15, 615.
[22]A. M. Baciewicz, C. R. Chrisman, C. K. Finch, T. H. Self, Curr. Med. Res. Opin. 2013, 29, 1.
[23]Tuberculosis 2008, 88, 145.
[24]Y. Crabol, E. Catherinot, N. Veziris, V. Jullien, O. Lortholary, J. Antimicrob. Chemother. 2016, 71, 1759.
[25]D. N. McMurray, Tuberculosis (Edinburgh, Scotland) 2003, 83, 131.
[26]J. Reynolds, S. K. Heysell, Expert Opin. Drug Metab. Toxicol. 2014, 10, 813.
[27]D. B. Aziz, J. L. Low, M. L. Wu, M. Gengenbacher, J. W. P. Teo, V. Dartois, T. Dick, Antimicrob Agents Ch 2017, 61.
[28]T. F. Blaschke, M. H. Skinner, Clin. Infect. Dis. 1996, 22 Suppl 1, S15.
[29]M. H. Skinner, T. F. Blaschke, Clin. Pharmacokinet. 1995, 28, 115.
[30]L. Wang, A. Wang, X. Zhao, X. Liu, D. Wang, F. Sun, Y. Li, Int. J. Pharm. 2012, 427, 284.
[31]B. Q. Li, M. Shan, X. Di, C. Gong, L. H. Zhang, Y. M. Wang, G. L. Wu, Rsc Adv 2017, 7, 30242.
[32]Y. Yuan, K. Choi, S. O. Choi, J. Kim, Rsc Adv 2018, 8, 19791.
[33]K. E. Samy, Y. Q. Cao, J. Kim, N. R. K. da Silva, A. Phone, M. M. Bloomer, R. B. Bhisitkul, T. A. Desai, J Ocul. Pharmacol. Ther. 2019, 35, 124.
[34]G. Ramachandran, V. Sudha, A. K. Hemanth Kumar, SAARC Journal of Tuberculosis, Lung Diseases and HIV/AIDS 2013, 9, 26.
[35]L. Solorio, D. Sundarapandiyan, A. Olear, A. A. Exner, J Pharm. Sci. 2015, 104, 3471.
[36]K. A. Hopkins, N. Vike, X. Li, J. Kennedy, E. Simmons, J. Rispoli, L. Solorio, Journal of Controlled Release 2019, 309, 289.
[37]Z. Ahmad, A. Shah, M. Siddiq, H.-B. Kraatz, RSC Adv. 2014, 4, 17028.
[38]C. Houga, J. Giermanska, S. Lecommandoux, R. Borsali, D. Taton, Y. Gnanou, J. F. Le Meins, Biomacromolecules 2009, 10, 32.
[39]L. Liu, K. Mao, W. T. Wang, H. C. Pan, F. Wang, M. Yang, H. Liu, AAPS PharmSciTech 2016, 17, 757.
[40]R. K. Thapa, F. Cazzador, K. G. Gronlien, H. H. Tonnesen, Colloids Surf B. Biointerfaces 2020, 195, 111250.
[41]H. M. Yapa, M. Boffito, A. Pozniak, J Acquir. Immune Defic. Syndr. 2016, 72, 138.
[42]S. Kouanda, H. G. Ouedraogo, K. Cisse, T. R. Compaore, G. Sulis, S. Diagbouga, A. Roggi, G. Tamagda, P. Villani, L. Sangare, J. Simpore, M. Regazzi, A. Matteelli, BMC Infect. Dis. 2020, 20, 449.
[43]Y. Fu, W. J. Kao, Expert opinion on drug delivery 2010, 7, 429.
[44]X. Wang, S. S. Venkatraman, F. Y. Boey, J. S. Loo, L. P. Tan, Biomaterials 2006, 27, 5588.
[45]S. Juvekar, H. Kathpalia, J. Control. Release 2017, 251, 75.
[46]R. P. Felix Lanao, A. M. Jonker, J. G. Wolke, J. A. Jansen, J. C. van Hest, S. C. Leeuwenburgh, Tissue engineering. Part B, Reviews 2013, 19, 380.
[47]D. Ma, A. J. McHugh, Int J Pharm 2010, 388, 1.
[48]X. Wang, D. J. Burgess, Adv Drug Deliv Rev 2021, 113912.
[49]L. Solorio, A. M. Olear, H. Zhou, A. C. Beiswenger, A. A. Exner, Drug Deliv Transl Res 2012, 2, 45.
[50]C. Hemandez, N. Gawlik, M. Goss, H. Zhou, S. Jeganathan, D. Gilbert, A. A. Exner, J. Control. Release 2016, 243, 225.
[51]R. B. Patel, L. Solorio, H. Wu, T. Krupka, A. A. Exner, J Control. Release 2010, 147, 350.
[52]A. Gopferich, Biomaterials 1996, 17, 103.
[53]R. B. Patel, A. N. Carlson, L. Solorio, A. A. Exner, J. Biomed. Mater. Res. A 2010, 94, 476.
[54]X. Zhang, L. Yang, C. Zhang, D. Liu, S. Meng, W. Zhang, S. Meng, Pharmaceutics 2019, 11.
[55]P. R. Ouyang, H. Dong, X. J. He, X. Cai, Y. B. Wang, J. L. Li, H. P. Li, Z. M. Jin, Mater Design 2019, 183.
[56]A. Gopferich, Biomaterials 1997, 18, 397.
[57]F. von Burkersroda, R. Gref, A. Gopferich, Biomaterials 1997, 18, 1599.
[58]J. M. Pean, M. C. Venier-Julienne, F. Boury, P. Menei, B. Denizot, J. P. Benoit, J. Control. Release 1998, 56, 175.
[59]M. Zare, H. Mobedi, J. Barzin, H. Mivehchi, A. Jamshidi, R. Mashayekhi, J Appl. Polym. Sci. 2008, 107, 3781.
[60]S. A. Montgomery, E. F. Young, P. G. Durham, K. E. Zulauf, L. Rank, B. K. Miller, J. D. Hayden, F. C. Lin, J. T. Welch, A. J. Hickey, M. Braunstein, PLoS One 2018, 13, e0204495.
[61] FDA, Inactive Ingredients Database, 2022, 2022
[62]N. Dookie, S. Rambaran, N. Padayatchi, S. Mahomed, K. Naidoo, J Antimicrob. Chemother. 2018, 73, 1138.
[63]G. Kaplan, J. Casoy, J. Zummo, Patient Prefer Adherence 2013, 7, 1171.
[64]S. Koocheki, S. S. Madaeni, P. Niroomandi, Saudi Pharm J 2011, 19, 255.
[65]J. A. Tamada, R. Langer, Proc. Natl. Acad. Sci. U.S.A 1993, 90, 552.
[66]F. von Burkersroda, L. Schedl, A. Gopferich, Biomaterials 2002, 23, 4221.
[67]H. K. Makadia, S. J. Siegel, Polymers (Basel) 2011, 3, 1377.
[68]S. Boimvaser, R. N. Mariano, L. N. Turino, J. R. Vega, Polym Bull 2016, 73, 209.
[69]B. Ji, C. Truffot-Pemot, C. Lacroix, M. C. Raviglione, R. J. O'Brien, P. Olliaro, G. Roscigno, J. Grosset, Am. Rev Respir. Dis. 1993, 148, 1541.
[70]D. Jabes, C. Della Bruna, R. Rossi, P. Olliaro, Antimicrob Agents Chemother 1994, 38, 2346.
[71]M. Strolin Benedetti, E. Pianezzola, G. Brianceschi, D. Jabes, C. Della Bruna, M. Rocchetti, I. Poggesi, J Antimicrob. Chemother. 1995, 36, 247.
[72] FDA, Guidance for Industry Estimating the Maximum Safe Starting Dose in Initial Clinical Trials for Therapeutics inAdult Healthy Volunteers, 2005,
[73]A. B. Nair, S. Jacob, Journal of basic and clinical pharmacy 2016, 7, 27.
[74] FDA, SUBLOCADE (buprenorphine), DrugApproval Package, 2017,
[75] FDA, Perseris (risperidone)extended release injectable 2018,
[76] FDA, FDA Approves Cabenuva and Vocabria for the Treatment of HIV-1 Infection, 2021, 2021
[77]J. Chen, K. Raymond, Ann. Clin. Microbiol. Antimicrob. 2006, 5, 3.

It will be understood that various details of the presently disclosed subject matter may be changed without departing from the scope of the presently disclosed subject matter. Furthermore, the foregoing description is for the purpose of illustration only, and not for the purpose of limitation.

1. An extended release or long-acting injectable composition, the composition comprising:
a drug or active agent;
a biocompatible solvent;
a biodegradable polymer; and
an amphiphilic additive, or a hydrophobic additive, or a combination of an amphiphilic additive and a hydrophobic additive.

2. The extended release or long-acting injectable composition of claim 1, wherein the extended release or long-acting injectable composition provides a sustained release of the drug or active agent upon administration to a subject in vivo or upon formation of an in-situ forming implant in vitro.

3. The extended release or long-acting injectable composition of any of claims 1 to 2, wherein the composition comprises the amphiphilic additive.

4. (canceled)

5. The extended release or long-acting injectable composition of any of claims 1 to 2, wherein the composition comprises the hydrophobic additive, wherein the hydrophobic additive increases release of the drug or active agent from a solidified implant formed from the extended release or long-acting injectable composition by at least 5%, optionally by about 20% to about 150%, as compared to a solidified implant without a hydrophobic additive.

6. (canceled)

7. The extended release or long-acting injectable composition of claim 5, wherein the hydrophobic additive comprises a fatty alcohol, wherein the fatty alcohol comprises hexacosanol, octacostanol, dotriacontanol, and/or combinations thereof.

8. The extended release or long-acting injectable composition of claim 5, wherein the hydrophobic additive comprises a terpene, wherein the terpene comprises nerolidol, farnesol, and/or combinations thereof.

9. The extended release or long-acting injectable composition of claim 5, wherein the hydrophobic additive comprises a sterols, wherein the sterol comprises cholesterol, sitosterol, stigmasterol, stigmastanol, and/or combinations thereof.

10. The extended release or long-acting injectable composition of claim 5, wherein the hydrophobic additive comprises a tocopherol, wherein the tocopherol comprises vitamin E, Vitamin E derivatives, and/or combinations thereof.

11. (canceled)

12. (canceled)

13. (canceled)

14. The extended release or long-acting injectable composition of any of claims 1 to 13, wherein the biocompatible solvent is selection from one or more of Dimethyl sulfoxide (DMSO), n-Methyl pyrrolidone (NMP), benzyl alcohol (BA), benzyl benzoate (BB) or combinations thereof.

15. The extended release or long-acting injectable composition of claim 14, wherein the biocompatible solvent comprises a cosolvent system using NMP and DMSO, wherein the DMSO:NMP ratio of about 1:99 to about 50:50, or wherein the DMSO:NMP ratio of about 1:99 to about 99:1.

16. The extended release or long-acting injectable composition of any of claims 1 to 15, wherein the biodegradable polymer comprises a low molecular weight (MW) polymer defined as a MW less than about 25 Da.

17. The extended release or long-acting injectable composition of any of claims 1 to 16, wherein the biodegradable polymer comprises a range of lactic acid:glycolic acid ratios of about 50:50 to about 95:5.

18. The extended release or long-acting injectable composition of any of claims 1 to 17, wherein the biodegradable polymer comprises a biodegradable poly(lactic-co5 glycolic-acid) (PLGA).

19. The extended release or long-acting injectable composition of any of claims 1 to 18, wherein the extended release or long acting injectable composition is configured as an in-situ forming implant (ISFI), wherein the extended release or long acting injectable composition comprises a liquid formulation that is configured to be injectable in a subject, wherein the extended release or long acting injectable composition is injectable subcutaneously.

20. The extended release or long-acting injectable composition of any of claims 1 to 19, wherein the extended release comprises a substantially sustained release of the drug or active agent over at least about 1 week.

21. The extended release or long-acting injectable composition of any of claims 1 to 20, wherein the composition comprises:
a biodegradable poly(lactic-co5 glycolic-acid) (PLGA), optionally wherein the PLGA is a polymer with molecular weight 10.6 kDa and lactic acid:glycolic acid ratio 50:50;
a biocompatible water miscible solvent;
an amphiphilic additive;
a hydrophobic additive; and
an active ingredient(s) or combination thereof selected from rifabutin (RFB), rifapentine, or rifampin, or any drug or drug combination for treating Nontuberculous Mycobacteria (NTM) and/or tuberculosis.

22. (canceled)

23. The extended release or long-acting injectable composition of any of claims 1 to 21, wherein the composition is configured to be administered to a subject in need of treatment as an in-situ forming implant (ISFI) about once a month.

24. The extended release or long-acting injectable composition of any of claims 1 to 21, wherein a concentration or quantity of the drug or active agent in the composition is increased by about 200% to about 350% as compared to a composition not having an amphiphilic additive.

25. A method of treating a subject, the method comprising administering to a subject in need of treatment an extended release or long-acting injectable composition of any of claims 1 to 24, wherein administration of the extended release or long-acting injectable composition to the subject forms a long-acting in-situ forming implant (LA ISFI) in the subject, wherein the subject is treated.

26. The method of claim 25, wherein the LA ISFI in the subject provides to the subject a drug or active agent for at least about 1 week or more.

27-50. (canceled)
